(19) United States
(12) Patent Application Publication
(10) Pub. No.: US 2006/0057127 A1
(43) Pub. Date: Mar. 16, 2006

(54) Cytokine-expressing cellular vaccines for treatment of prostate cancer

(76) Inventors: Pocheng Liu, Seattle, WA (US); Kuoting Anthony Wu, Mountain View, CA (US); Johanna Sy, Pacifica, CA (US); Flavia Borellini, Foster City, CA (US)

Correspondence Address: Supervisor, Patent Prosecution Services;Piper Rudnick LLP, 1200 Nineteenth Street, N.W., Washington, DC, 20036-2412, US

(21) Appl. No.: 10/937,658

(22) Filed: Sep. 10, 2004

(51) Int. Cl. A61K 48/00

(52) U.S. Cl. 424/93.21; 514/44

(57) ABSTRACT

Genetically modified cytokine-expressing cells for use as vaccines in the treatment of prostate cancer are provided. More specifically, genetically modified, GM-CSF expressing cells as a means to generate an enhanced immune response to beta filamin and the use thereof in the treatment of prostate cancer are described.

### BACKGROUND OF THE INVENTION

1. Field of the Invention

The present invention relates to genetically modified cytokine-expressing cells for use as vaccines for the treatment of prostate cancer. More specifically, the present invention relates to identification of an enhanced immune response to an antigen, beta filamin, which is detected following administration of genetically modified GM-CSF expressing cells to a prostate cancer patient.

2. Background of the Technology

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

In a functioning immune system, antigens are processed and expressed on the cell surface in the context of major histocompatibility complex (MHC) class I and II molecules. When complexed to antigens, the MHC class I and II molecules are recognized by CD8+ and CD4+ T cells, respectively. This recognition generates a set of secondary cellular signals and the paracrine release of specific cytokines, that mediate interactions between cells and stimulate host defenses to fight off disease. The release of cytokines then results in the proliferation of antigen-specific immune cells.

Numerous cytokines have been shown to play a role in regulation of the immune response to tumors. For example, U.S. Pat. No. 5,098,702 describes use of combinations of TNF, IL-2 and IFN-beta in synergistically effective amounts to combat existing tumors. U.S. Pat. Nos. 5,078,996, 5,637,483 and 5,904,920 describe the use of GM-CSF for treatment of tumors. However, direct administration of cytokines for cancer therapy may not be practical, as they are often systemically toxic. (See, for example, Asher et al., J. Immunol. 146:3227-3234, 1991 and Havell et al., J. Exp. Med. 167:1067-1085, 1988.)

An expansion of this approach involves the use of genetically modified tumor cells which express cytokines locally at the vaccine site. Activity has been demonstrated in tumor models using a variety of immunomodulatory cytokines, including IL-4, IL-2, TNF-alpha, G-CSF, IL-7, IL-6 and GM-CSF, as described in Golumbeck PT et al., Science 254:13-716, 1991; Gansbacher B et al., J. Exp. Med. 172:1217-1224, 1990; Fearon ER et al., Cell 60:397-403, 1990; Gansbacher B et al., Cancer Res. 50:7820-25, 1990; Teng M et al., PNAS 88:3535-3539, 1991; Columbo MP et al., J. Exp. Med. 174:1291-1298, 1991; Aoki et al., Proc Natl Acad Sci USA. 89(9):3850-4, 1992; Porgador A, et al., Nat Immun. 13(2-3):113-30, 1994; DranoffG et al., PNAS 90:3539-3543, 1993; Lee C T et al., Human Gene Therapy 8:187-193, 1997; Nagai E et al., Cancer Immunol. Immonther. 47:2-80, 1998 and Chang A et al., Human Gene Therapy 11:839-850, 2000, respectively. The use of autologous cancer cells as vaccines to augment anti-tumor immunity has been explored for some time. See, e.g., Oettgen et al., “The History of Cancer Immunotherapy”, In: Biologic Therapy of Cancer, Devita et al. (eds.) J. Lippincot Co., pp 87-199, 1991; Armstrong T D and Jaffee E M, Surg Oncol Clin N Am. 11(3):681-96, 2002; and Bodey B et al., Anticancer Res 20(4):2665-76, 2000).

Several phase I/II human trials using GM-CSF-secreting autologous or allogeneic tumor cell vaccines have been performed (Simons et al. Cancer Res 1999 59:5160-8; Soiffer et al. Proc Natl Acad Sci USA 1998 95:13141-6; Simons et al. Cancer Res 1997 57:1537-46; Jaffee et al. J Clin Oncol 2001 19:145-56; Salgia et al. J Clin Oncol 2003 21:624-30; Soiffer et al. J Clin Oncol 2003 21:3343-50; Nemunaitis et al. J Natl Cancer Inst. 2004 Feb. 18 96(4):326-31; Borello and Pardoll, Growth Factor Rev. 13(2):185-93, 2002; and Thomas et al., J. Exp. Med. 200(3)297-306, 2004).

Following administration of genetically modified GM-CSF-expressing cancer cells to a patient an enhanced immune response has been shown to result and preliminary clinical efficacy against prostate and other cancers has been demonstrated in Phase I/II clinical trails. However, there remains a need for improved strategies involving the use of cellular vaccines for use in treatment of prostate cancer.

### SUMMARY OF THE INVENTION

The present invention provides compositions and methods for treating prostate cancer in a subject, comprising genetically modified cytokine-expressing cells. In one aspect, the invention includes a method of treating prostate cancer in a subject, by administering genetically modified cytokine-expressing cells to the subject for treatment of prostate cancer.

The method is carried out by genetically modifying (transducing) a first population of tumor cells to produce a cytokine, e.g., GM-CSF, and administering the first population of tumor cells alone or in combination with a second population of tumor cells to the subject. Following administration of the genetically modified cytokine-expressing cells, an immune response to an approximately 278 kD antigen as determined by SDS-PAGE is detected, wherein the immune response is not detected prior to administering the cytokine-expressing cells. The approximately 278 kD antigen was identified as beta filamin.

The tumor cells may be tumor cells from the same individual (autologous), from a different individual (allogeneic) or bystander cells and are typically rendered proliferation-incompetent prior to administration. Typically, the tumor cells are of the same type as the tumor or cancer being treated, e.g., the genetically modified cytokine-expressing cells are prostate or prostate cancer cells (e.g., PC-3 cells or LNCaP cells) and the subject has prostate cancer. The immune response may be a humoral or cellular immune response.

Preferably following administration of the cytokine-expressing cells to a prostate cancer patient, an improved therapeutic outcome for the patient is evident.

### BRIEF DESCRIPTION OF THE FIGURES

The foregoing and other objects of the present invention, the various features thereof, as well as the invention itself, may be more fully understood from the following description, when read together with the accompanying drawings.

FIGS. 1A-D are a schematic representation of MFG vectors containing a cytokine-encoding sequence useful in the methods and vaccines of the present invention.

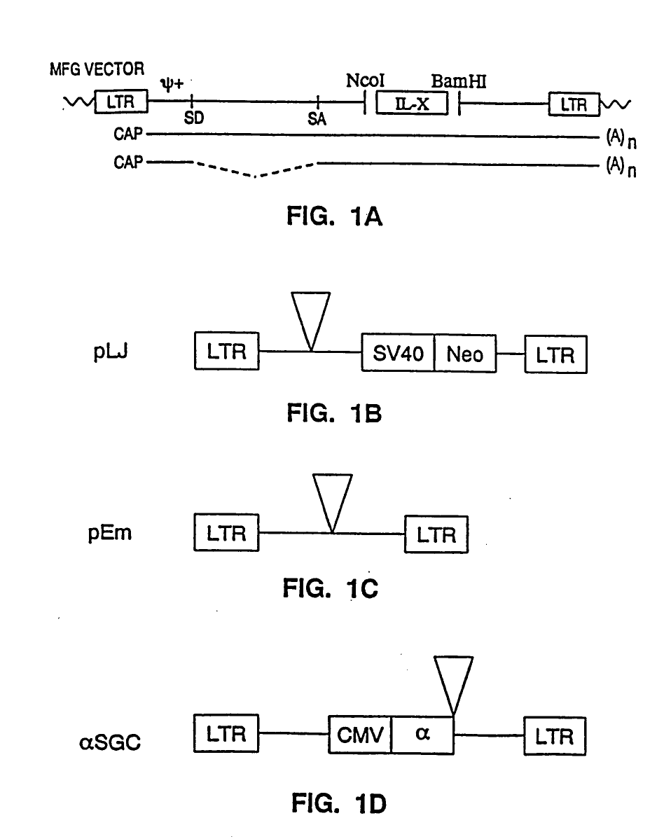

FIG. 1E is a schematic representation of a GM-CSF-encoding adenovirus vector (AV-GM-CSF) useful in methods and vaccines of the present invention.

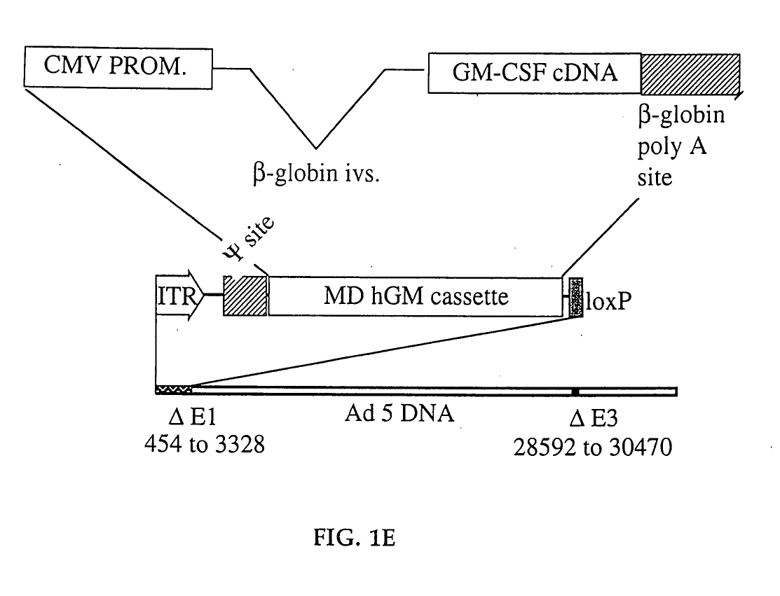

FIG. 1F is a schematic representation of a recombinant adeno-associated viral (AAV) vector plasmid (SSV9/MD2-hGM) useful in methods and vaccines of the present invention.

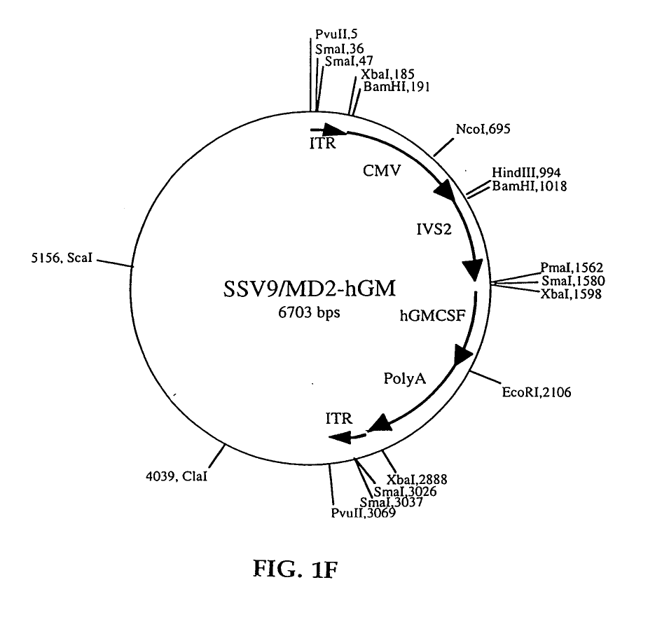

FIG. 1G is a schematic representation of a recombinant lentivirus vector containing a GM-CSF expression cassette flanked by HIV LTRs, useful in the methods and vaccines of the present invention.

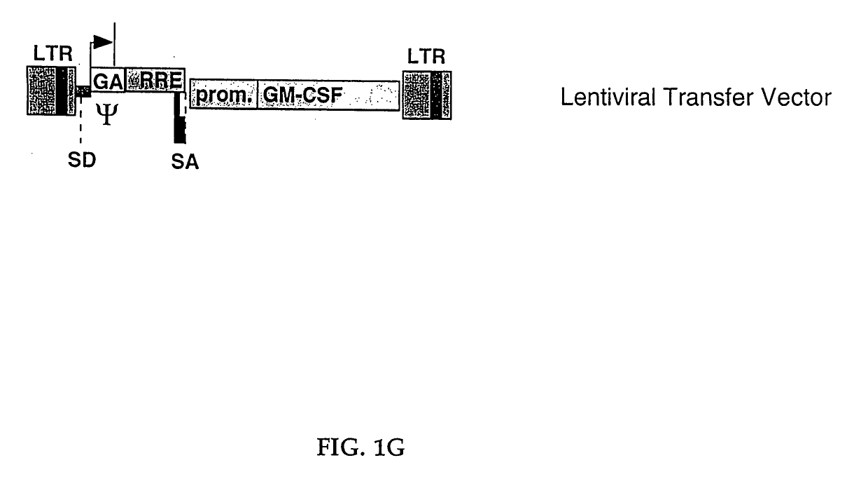

FIG. 1H is a schematic representation of an HSV-1-based vector containing a GM-CSF expression cassette replacing the ICP22 HSV gene, useful in the methods and vaccines of the present invention.

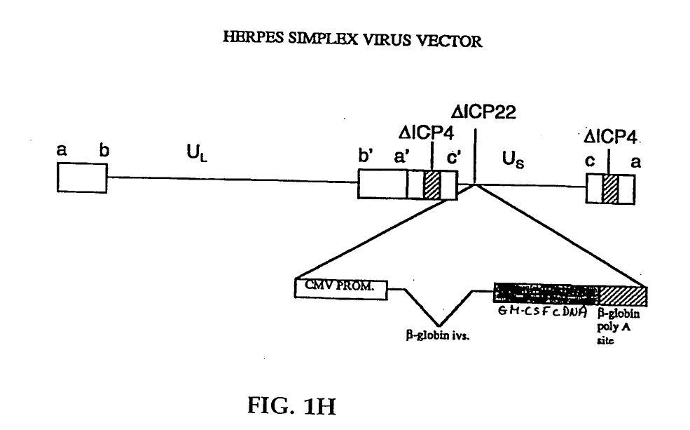

FIG. 1I is a schematic representation of an SV-40-based plasmid (pSV HD GM-CSFII) including a GM-CSF expression cassette, the SV-40 origin of replication, and viral late genes, useful in the methods and vaccines of the present invention.

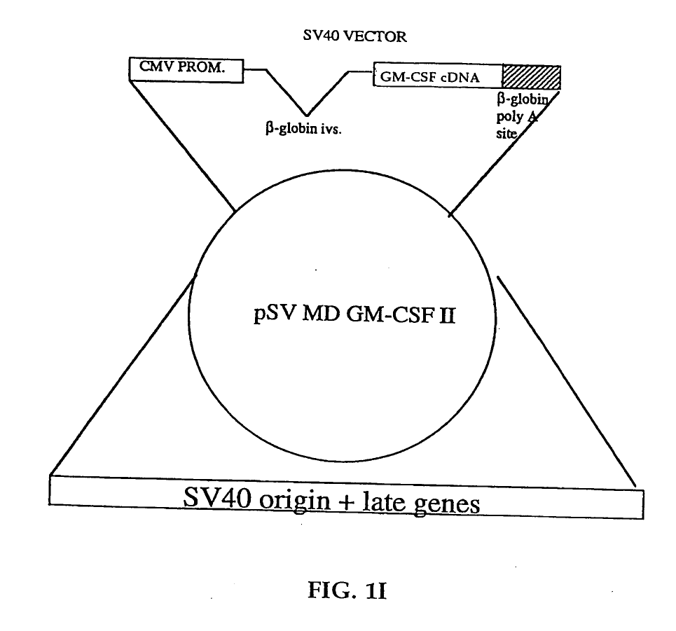

FIG. 1J is a schematic representation of a vaccinia virus expression cassette including a vaccinia virus promoter and termination sequence, useful in the methods and vaccines of the present invention.

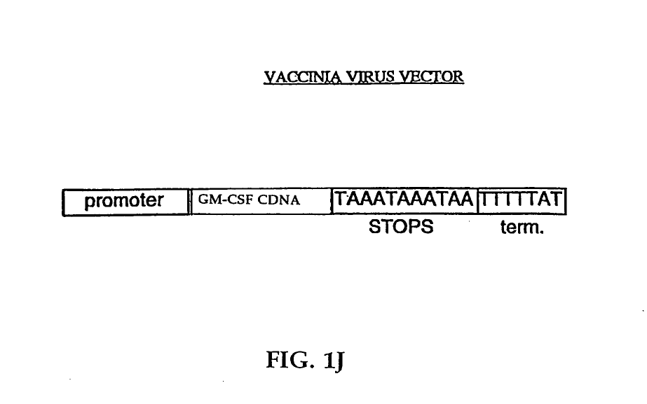

FIG. 2 shows the complete PSA response of patient 804 in an allogeneic prostate GVAX® clinical trial. The initial dose of the vaccine treatment was administered on day 0.

FIG. 3 represents the results of a Western blot analysis which shows that post-vaccination serum from patient 804 was found to recognize an antigen migrating at approximately 278 kD that is present in PC-3 but not LNCaP cells.

FIG. 4 represents the results of a Western blot analysis using pre-vaccination (Wk 0) and post-vaccination (Wk 24) sera from patient 804, which shows that post-vaccination serum from patient 804 was found to recognize an approximately 278 kD antigen that was also detected in primary normal prostate epithelial, prostate stromal, and prostate smooth muscle cell lines, PrEC, PrSC, and PrSmC, respectively, but at lower levels than in PC-3.

FIG. 5 represents the results of a Western blot analysis using pre-vaccination (Wk 0) and post-vaccination (Wk 24) sera from patient 804, which shows that post-vaccination serum from patient 804 also recognizes an approximately 278 kD antigen that is over expressed in the non-small cell lung carcinoma (NSCLC) H157, but not expressed in H1395 NSCLC adenocarcinoma cell line.

FIG. 6 represents the results of a Western blot analysis using rabbit anti-human filamin monoclonal antibodies from Chemicon International (Temecula, Calif.) which shows reactivity with an approximately 278 kD protein expressed in PC-3 cells but not LNCAP cells.

### DETAILED DESCRIPTION OF THE INVENTION

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Anand, 1992, Techniques for the Analysis of Complex Genomes, Academic Press, New York; Guthrie and Fink, 1991, Guide to Yeast Genetics and Molecular Biology, Academic Press, New York; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

Definitions

Unless otherwise indicated, all terms used herein have the same meaning as they would to one skilled in the art.

The publications and other materials including all patents, patent applications, publications (including published patent applications), and database accession numbers referred to in this specification are used herein to illuminate the background of the invention and in particular, cases to provide additional details respecting the practice. The publications and other materials including all patents, patent applications, publications (including published patent applications), and database accession numbers referred to in this specification are incorporated herein by reference to the same extent as if each were specifically and individually indicated to be incorporated by reference in its entirety.

In describing the present invention, the following terms are employed and are intended to be defined as indicated below.

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G).

The terms “coding sequence” and “coding region” refer to a nucleic acid sequence that is transcribed into RNA such as mRNA, rRNA, tRNA, snRNA, sense RNA or antisense RNA. In one embodiment, the RNA is then translated in a cell to produce a protein.

The term “ORF” means Open Reading Frame.

The term “gene” refers to a defined region that is located within a genome and that, in addition to the aforementioned coding sequence, comprises other, primarily regulatory, nucleic acid sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The terms “heterologous” and “exogenous” as used herein with reference to nucleic acid molecules such as promoters and gene coding sequences, refer to sequences that originate from a source foreign to a particular vector or host cell or, if from the same source, are modified from their original form. Thus, a heterologous gene in a virus or cell includes a gene that is endogenous to the particular virus or cell but has been modified through, for example, codon optimization. The terms “heterologous” and “exogenous” may also be used with reference to non-naturally occurring multiple copies of a naturally occurring nucleic acid sequence. Thus, the terms refer to a nucleic acid segment that is foreign or heterologous to the virus or cell, or homologous to the virus or cell but in a position within the host viral or cellular genome other than that in which it is ordinarily found.

The term “homologous” as used herein with reference to a nucleic acid molecule refers to a nucleic acid sequence naturally associated with a host virus or cell.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “native” refers to a gene or protein that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleic acid molecule.

“Regulatory elements” are sequences involved in controlling the expression of a nucleotide sequence. Regulatory elements include promoters, enhancers, and termination signals. They also typically encompass sequences required for proper translation of the nucleotide sequence.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

The term “expression” refers to the transcription and/or translation of an endogenous gene, transgene or coding region in a cell. In the case of an antisense construct, expression may refer to the transcription of the antisense DNA only.

The term “up-regulated” as used herein means that a greater quantity of the RNA for a specific gene can be detected in the target cell as compared to another cell. For example, if a tumor cell that produces more telomerase RNA as compared to a non-tumor cell, the tumor cell has up-regulated expression of telomerase. Expression is considered up regulated when the quantity of specific RNA in a target cell (e.g. tumor cell) is at least 3-fold greater than in another cell (non-tumor cell). In another embodiment, the quantity of specific RNA is at least 5-fold greater. In another embodiment, the quantity of specific RNA is at least 10-fold greater using a technique routinely employed by those skilled in the (e.g. Northern Blot Assay).

The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art. The vectors utilized in the present invention may optionally code for a selectable marker.

As used herein, the term “viral vector” is used according to its art-recognized meaning. It refers to a nucleic acid vector construct that includes at least one element of viral origin and may be packaged into a viral vector particle. The viral vector particles may be utilized for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno-associated viral (AAV) vectors. Suitable viral vectors are described in U.S. Pat. Nos. 6,057,155, 5,543,328 and 5,756,086, expressly incorporated by refernce herein.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo.

A nucleic acid sequence is “operatively linked” when it is placed into a functional relationship with another nucleic acid sequence. For example, a promoter or regulatory DNA sequence is said to be “operatively linked” to a DNA sequence that codes for an RNA or a protein if the two sequences are operatively linked, or situated such that the promoter or regulatory DNA sequence affects the expression level of the coding or structural DNA sequence. Operatively linked DNA sequences are typically, but not necessarily, contiguous.

A “selectable marker” is a protein whose expression in a cell gives the cell a selective advantage. The selective advantage possessed by the cells transformed with the selectable marker gene may be due to their ability to grow in the presence of a negative selective agent, such as an antibiotic, compared to the growth of non-transduced cells. The selective advantage possessed by the transformed cells, compared to non-transduced cells, may also be due to their enhanced or novel capacity to utilize an added compound as a nutrient, growth factor or energy source. Selective marker proteins include those that allow detection of the transduced cells and possibly their separation from non-transduced cells. For example, Green Fluorescent Protein (GFP) can be used as a selectable marker. In one embodiment, cells are transduced with a vector encoding both a beta-filamin or immunogenic fragment thereof and a GFP protein. The transduced cells expressing GFP are separated using fluorescence-activated cell sorting (FACS). The selectable marker protein can allow for transduced cells to be mostly separated from non-transduced cells. One skilled in the art recognizes that selection and separation techniques are not usually 100% and that small percentages of a population of unselected cells are acceptable for the present invention.

The term “consists essentially of” or “consisting essentially of” as used herein with reference to a particular nucleotide sequence means that the particular sequence may have additional residues on either the 5′ or 3′ end or both, wherein the additional residues do not materially affect the basic and novel characteristics of the recited sequence.

By the term “transduction” is meant the introduction of an exogenous nucleic acid into a cell by physical means. For example, transduction includes the introduction of exogenous nucleic acid into a cell using a viral particle of the invention. For various techniques for manipulating mammalian cells, see Keown et al., Methods of Enzymology 185: 527-537 (1990).

As used herein, a “packaging cell” is a cell that is able to package viral genomes or modified genomes to produce viral particles. It can provide a missing gene product or its equivalent. Thus, packaging cells can provide complementing functions for the genes deleted in an viral genome and are able to package the viral genomes into virus particles. The production of such particles requires that the genome be replicated and that those proteins necessary for assembling an infectious virus are produced. The particles also can require certain proteins necessary for the maturation of the viral particle. Such proteins can be provided by the vector or by the packaging cell.

As used herein, a “retroviral transfer vector” refers to an expression vector that comprises a nucleotide sequence that encodes a transgene and that further comprises nucleotide sequences necessary for packaging of the vector. Preferably, the retroviral transfer vector also comprises the necessary sequences for expressing the transgene in cells.

As used herein, a “second generation” lentiviral vector system refers to a lentiviral packaging system that lacks functional accessory genes, such as one from which the accessory genes, vif, vpr, vpu and nef, have been deleted or inactivated. See, e.g., Zufferey et al., 1997, Nat. Biotechnol. 15:871-875.

As used herein, a “third generation” lentiviral vector system refers to a lentiviral packaging system that has the characteristics of a second generation vector system, and that further lacks a functional tat gene, such as one from which the tat gene has been deleted or inactivated. Typically, the gene encoding rev is provided on a separate expression construct. See, e.g., Dull et al., 1998, J. Virol. 72(11):8463-8471.

As used herein, “pseudotyped” refers to the replacement of a native envelope protein with a heterologous or functionally modified envelope protein.

The term “exposing”, as used herein means bringing a transgene-encoding vector in contact with a target cell. Such “exposing”, may take place in vitro, ex vivo or in vivo.

As used herein, the terms “stably transformed”, “stably transfected” and “transgenic” refer to cells that have a non-native (heterologous) nucleic acid sequence integrated into the genome. Stable transformation is demonstrated by the establishment of cell lines or clones comprised of a population of daughter cells containing the transfecting DNA. In some cases, “transformation” is not stable, i.e., it is transient. In the case of transient transformation, the exogenous or heterologous DNA is expressed, however, the introduced sequence is not integrated into the genome.

By the term “cytokine” or grammatical equivalents, herein is meant the general class of hormones of the cells of the immune system, including lymphokines, monokines, and others. The definition includes, without limitation, those hormones that act locally and do not circulate in the blood, and which, when used in accord with the present invention, will result in an alteration of an individual's immune response. The term “cytokine” or “cytokines” as used herein refers to the general class of biological molecules, which affect cells of the immune system. The definition is meant to include, but is not limited to, those biological molecules that act locally or may circulate in the blood, and which, when used in the compositions or methods of the present invention serve to regulate or modulate an individual's immune response to cancer. Exemplary cytokines for use in practicing the invention include, but are not limited to, IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, ICAM, macrophage colony stimulating factor (M-CSF), granulocyte colony stimulating factor (G-CSF) and granulocyte-macrophage colony stimulating factor (GM-CSF).

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, New York. Generally, highly stringent hybridization and wash conditions are selected to be about 5° C. to 20° C. (preferably 5° C.) lower than the thermal melting point (Tₘ) for the specific sequence at a defined ionic strength and pH. Typically, under highly stringent conditions a probe will hybridize to its target subsequence, but to no other unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein or by visual inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

A “normal cell status” or “normal physiological state” is the status of a cell which exists in normal physiological conditions and which is non-dividing or divides in a regulated manner, i.e., a cell in a normal physiological state. An “aberrant cell status” is defined in relation to a cell of the same type, which is in a non-dividing/regulated dividing state and under normal physiological conditions. It follows that a cell which has an “aberrant cell status” exhibits unregulated cell division.

As used herein, the terms “cancer”, “cancer cells”, “neoplastic cells”, “neoplasia”, “tumor”, and “tumor cells” (used interchangeably) refer to cells that exhibit relatively autonomous growth, so that they exhibit an aberrant growth phenotype or aberrant cell status characterized by a significant loss of control of cell proliferation. A tumor cell may be a hyperplastic cell, a cell that shows a lack of contact inhibition of growth in vitro or in vivo, a cell that is incapable of metastasis in vivo, or a cell that is capable of metastasis in vivo. Neoplastic cells can be malignant or benign. It follows that cancer cells are considered to have an aberrant cell status. “Tumor cells” may be derived from a primary tumor or derived from a tumor metastases. The “tumor cells” may be recently isolated from a patient (a “primary tumor cell”) or may be the product of long term in vitro culture.

The term “primary tumor cell” is used in accordance with the meaning in the art. A primary tumor cell is a cancer cell that is isolated from a tumor in a mammal and has not been extensively cultured in vitro.

The term “antigen from a tumor cell” and “tumor antigen” and “tumor cell antigen” may be used interchangeably herein and refer to any protein, peptide, carbohydrate or other component derived from or expressed by a tumor cell which is capable of eliciting an immune response. The definition is meant to include, but is not limited to, whole tumor cells, tumor cell fragments, plasma membranes taken from a tumor cell, proteins purified from the cell surface or membrane of a tumor cell, unique carbohydrate moieties associated with the cell surface of a tumor cell or tumor antigens expressed from a vector in a cell. The definition also includes those antigens from the surface of the cell, which require special treatment of the cells to access.

The term “genetically modified tumor cell” as used herein refers to a composition comprising a population of cells that has been genetically modified to express a transgene, and that is administered to a patient as part of a cancer treatment regimen. The genetically modified tumor cell vaccine comprises tumor cells which are “autologous” or “allogeneic” to the patient undergoing treatment or “bystander cells” that are mixed with tumor cells taken from the patient. A GM-CSF-expressing genetically modified tumor cell vaccine may be referred to herein as “GVAX”®. Autologous and allogeneic cancer cells that have been genetically modified to express a cytokine, e.g., GM-CSF, followed by readministration to a patient for the treatment of cancer are described in U.S. Pat. Nos. 5,637,483, 5,904,920, 6,277,368 and 6,350,445, each of which is expressly incorporated by reference herein. A form of GM-CSF-expressing genetically modified cancer cells or a “cytokine-expressing cellular vaccine” for the treatment of pancreatic cancer is described in U.S. Pat. Nos. 6,033,674 and 5,985,290, both of which are expressly incorporated by reference herein. A universal immunomodulatory cytokine-expressing bystander cell line is described in U.S. Pat. No. 6,464,973, expressly incorporated by reference herein.

The term “enhanced expression” as used herein, refers to a cell producing higher levels of a particular protein than would be produced by the naturally occurring cell or the parental cell from which it was derived. Cells may be genetically modified to increase the expression of a cytokine, such as GM-CSF, or an antigen the immune response to which is enhanced following administration of a cytokine-expressing cellular vaccine, such as GVAX®. The expression of an endogenous antigen may be increased using any method known in the art, such as genetically modifying promoter regions of genomic sequences or genetically altering cellular signaling pathways to increase production of the antigen. Also, cells can be transduced with a vector coding for the antigen or immunogenic fragment thereof.

By the term “systemic immune response” or grammatical equivalents herein is meant an immune response which is not localized, but affects the individual as a whole, thus allowing specific subsequent responses to the same stimulus.

As used herein, the term “proliferation-incompetent” or “inactivated” refers to cells that are unable to undergo multiple rounds of mitosis, but still retain the capability to express proteins such as cytokines or tumor antigens. This may be achieved through numerous methods known to those skilled in the art. Embodiments of the invention include, but are not limited to, treatments that inhibit at least about 95%, at least about 99% or substantially 100% of the cells from further proliferation. In one embodiment, the cells are irradiated at a dose of from about 50 to about 200 rads/min or from about 120 to about 140 rads/min prior to administration to the mammal. Typically, when using irradiation, the levels required are 2,500 rads, 5,000 rads, 10,000 rads, 15,000 rads or 20,000 rads. In several embodiments of the invention the cells produce beta-filamin or immunogenic fragment thereof, two days after irradiation, at a rate that is at least about 10%, at least about 20%, at least about 50% or at least about 100% of the pre-irradiated level, when standardized for viable cell number. In one embodiment of the invention, cells are rendered proliferation incompetent by irradiation prior to administration to the subject.

By the term “individual”, “subject” or grammatical equivalents thereof is meant any one individual mammal.

By the term “reversal of an established tumor” or grammatical equivalents herein is meant the suppression, regression, or partial or complete disappearance of a pre-existing tumor. The definition is meant to include any diminution in the size, potency or growth rate of a pre-existing tumor.

The terms “treatment”, “therapeutic use”, or “medicinal use” as used herein, shall refer to any and all uses of the claimed compositions which remedy a disease state or symptom, or otherwise prevent, hinder, retard, or reverse the progression of disease or other undesirable symptoms in any way whatsoever.

The term “administered” refers to any method that introduces the cells of the invention (e.g. cancer vaccine) to a mammal. This includes, but is not limited to, intradermal, parenteral, intramuscular, subcutaneous, intraperitoneal, intranasal, intravenous (including via an indwelling catheter), intratumoral, via an afferent lymph vessel, or by another route that is suitable in view of the patient's condition. The compositions of this invention may be administered to the subject at any site. For example, they can be delivered to a site that is “distal” to or “distant” from the primary tumor.

The term “increased immune response” as used herein means that a detectable increase of a specific immune activation is detectable (e.g. an increase in B-cell and/or T-cell response). An example of an increased immune response is an increase in the amount of an antibody that binds an antigen which is not detected or is detected a lower level prior to administration of a cytokine-expressing cellular vaccine of the invention. Another example, is an increased cellular immune response. A cellular immune response involves T cells, and can be observed in vitro (e.g. measured by a Chromium release assay) or in vivo. An increased immune response is typically accompanied by an increase of a specific population of immune cells.

By the term “retarding the growth of a tumor” is meant the slowing of the growth rate of a tumor, the inhibition of an increase in tumor size or tumor cell number, or the reduction in tumor cell number, tumor size, or numbers of tumors.

The term “inhibiting tumor growth” refers to any measurable decrease in tumor mass, tumor volume, amount of tumor cells or growth rate of the tumor. Measurable decreases in tumor mass can be detected by numerous methods known to those skilled in the art. These include direct measurement of accessible tumors, counting of tumor cells (e.g. present in blood), measurements of tumor antigens (e.g. Prostate Specific Antigen (PSA), Alphafetoprotein (AFP) and various visualization techniques (e.g. MRI, CAT-scan and X-rays). Decreases in the tumor growth rate typically correlates with longer survival time for a mammal with cancer.

By the term “therapeutically effective amount” or grammatical equivalents herein refers to an amount of an agent, e.g., a cytokine-expressing cellular vaccine of the invention, that is sufficient to modulate, either by stimulation or suppression, the immune response of an individual. This amount may be different for different individuals, different tumor types, and different preparations. The “therapeutically effective amount” is determined using procedures routinely employed by those of skill in the art such that an “improved therapeutic outcome” results.

As used herein, the terms “improved therapeutic outcome” and “enhanced therapeutic efficacy”, relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including an increase in life expectancy or an improvement in quality of life (as further described herein).

By the terms “inactivated cells” and “proliferation-incompetent cells” or grammatical equivalents herein are meant cells inactivated by treatment rendering them proliferation-incompetent. This treatment results in cells which are unable to undergo multiple rounds of mitosis, but still retain the capability to express proteins such as cytokines and/or tumor antigens. This may be achieved through numerous methods known to those skilled in the art. An “irradiated cell” is one example of such an inactivated cell. Such irradiated cells have been exposed to sufficient irradiation to render them proliferation-incompetent.

Cellular Vaccine Compositions of the Invention

The present invention relates to a method of treating prostate cancer in a subject, by administering genetically modified cytokine-expressing cells to the subject as part of a therapeutic treatment for cancer. The method is carried out by genetically modifying (transducing) a first population of tumor cells to produce a cytokine, e.g., GM-CSF, and administering the first population of tumor cells alone or in combination with a second population of tumor cells to the subject such that following administration an immune response to an approximately 278 kD antigen as determined by SDS-PAGE is detected, wherein the immune response is not detected prior to administering the cytokine-expressing cells. The tumor cells may be tumor cells from the same individual (autologous), from a different individual (allogeneic) or bystander cells (further described below). Typically, the tumor cells are from a tumor cell line of the same type as the tumor or cancer being treated, e.g., the modified cells are prostate or prostate cancer cells and the patient has prostate cancer. The approximately 278 kD antigen has been identified as beta filamin.

In one aspect of the invention, the immune response is a humoral immune response. Typically the genetically modified tumor cells are rendered proliferation incompetent prior to administration. In one embodiment, the mammal is a human who harbors prostate tumor cells of the same type as the genetically modified cytokine-expressing tumor cells. In a preferred embodiment, an improved therapeutic outcome is evident following administration of the genetically modified cytokine-expressing tumor cells to the subject. Any of the various parameters of an improved therapeutic outcome for a prostate cancer patient known to those of skill in the art may be used to assess the efficacy of genetically modified cytokine-expressing tumor cell therapy, e.g., a reduction in the serum level of PSA.

In still another aspect, the invention provides a method for stimulating a systemic immune response in a prostate cancer patient, by administering a therapeutically effective amount of proliferation incompetent genetically modified cytokine-expressing cells to the subject. The systemic immune response to the tumor may result in tumor regression or inhibit the growth of the tumor.

In one preferred embodiment of the invention, a viral or nonviral vector is utilized to deliver a human GM-CSF transgene (coding sequence) to a human tumor cell ex vivo. After transduction, the cells are irradiated to render them proliferation incompetent. The proliferation incompetent GM-CSF expressing tumor cells are then re-administered to the patient (e.g., by the intradermal or subcutaneous route) and thereby function as a cancer vaccine. The human tumor cell may be a primary tumor cell or derived from a tumor cell line.

In general, the genetically modified tumor cells for use in practicing the invention include one or more of autologous tumor cells, allogeneic tumor cells and tumor cell lines (i.e., bystander cells). The tumor cells may be transduced in vitro, ex vivo or in vivo. Autologous and allogeneic cancer cells that have been genetically modified to express a cytokine, e.g., GM-CSF, followed by readministration to a patient for the treatment of cancer are described in U.S. Pat. Nos. 5,637,483, 5,904,920 and 6,350,445, expressly incorporated by reference herein. A form of GM-CSF-expressing genetically modified tumor cells or a “cytokine-expressing cellular vaccine” (“GVAX”®), for the treatment of pancreatic cancer is described in U.S. Pat. Nos. 6,033,674 and 5,985,290, expressly incorporated by reference herein. A universal immunomodulatory genetically modified bystander cell line is described in U.S. Pat. No. 6,464,973, expressly incorporated by reference herein.

An allogeneic form of GVAX® wherein the cellular vaccine comprises one or more prostate tumor cell lines selected from the group consisting of DU145, PC-3, and LNCaP is described in WO/0026676, expressly incorporated by reference herein. LNCaP is a PSA-producing prostate tumor cell line, while PC-3 and DU-145 are non-PSA-producing prostate tumor cell lines (Pang S. et al., Hum Gene Ther. 1995 November; 6(11):1417-1426).

Clinical trials employing GM-CSF-expressing cellular vaccines (GVAX®) have been undertaken for treatment of prostate cancer, melanoma, lung cancer, pancreatic cancer, renal cancer, and multiple myeloma. A number of clinical trials using GVAX® cellular vaccines have been described, most notably in melanoma, and prostate, renal and pancreatic carcinoma (Simons J W et al. Cancer Res. 1999; 59:5160-5168; Simons J W et al. Cancer Res 1997; 57:1537-1546; Soiffer R et al. Proc. Natl. Acad. Sci USA 1998; 95:13141-13146; Jaffee, et al. J Clin Oncol 2001; 19:145-156; Salgia et al. J Clin Oncol 2003 21:624-30; Soiffer et al. J Clin Oncol 2003 21:3343-50; Nemunaitis et al. J Natl Cancer Inst. 2004 Feb. 18 96(4):326-31).

By way of example, in one approach, genetically modified GM-CSF expressing tumor cells are provided as an allogeneic or bystander cell line and one or more additional cancer therapeutic agents is included in the treatment regimen. In another approach, one or more additional transgenes are expressed by an allogeneic or bystander cell line while a cytokine (i.e., GM-CSF) is expressed by autologous or allogeneic cells. The GM-CSF coding sequence is introduced into the tumor cells using a viral or non-viral vector and routine methods commonly employed by those of skill in the art. The preferred coding sequence for GM-CSF is the genomic sequence described in Huebner K. et al., Science 230(4731):1282-5,1985, however, in some cases the cDNA form of GM-CSF finds utility in practicing the invention (Cantrell et al., Proc. Natl. Acad. Sci., 82, 6250-6254, 1985).

In general, the genetically modified tumor cells are cryopreserved prior to administration. Preferably, the genetically modified tumor cells are irradiated at a dose of from about 50 to about 200 rads/min, even more preferably, from about 120 to about 140 rads/min prior to administration to the patient. Preferably, the cells are irradiated with a total dose sufficient to inhibit substantially 100% of the cells, from further proliferation. Thus, desirably the cells are irradiated with a total dose of from about 10,000 to 20,000 rads, optimally, with about 15,000 rads. Typically more than one administration of cytokine (e.g., GM-CSF) producing cells is delivered to the subject in a course of treatment. Dependent upon the particular course of treatment, multiple injections may be given at a single time point with the treatment repeated at various time intervals. For example, an initial or “priming” treatment may be followed by one or more “booster” treatments. Such “priming” and “booster” treatments are typically delivered by the same route of administration and/or at about the same site. When multiple doses are administered, the first immunization dose may be higher than subsequent immunization doses. For example, a 5×10⁶ prime dose may be followed by several booster doses of 10⁶ to 3×10⁶ GM-CSF producing cells.

A single injection of cytokine-producing cells is typically between about 10⁶ to 10⁸ cells, e.g., 1×10⁶, 2×10⁶, 3×10⁶, 4×10⁶, 5×10⁶, 6×10⁶, 7×10⁶, 8×10⁶, 9×10⁶, 10⁷, 2×10⁷, 5×10⁷, or as many as 10⁸ cells. In one embodiment, there are between 10⁶ and 10⁸ cytokine-producing cells per unit dose. The number of cytokine-producing cells may be adjusted according to the level of cytokine produced by a given cytokine producing cellular vaccine.

Embodiments of the invention include, but are not limited to cytokine-producing cells in a dose that are capable of producing at least 500 ng of GM-CSF per 24 hours per one million cells. Determination of optimal cell dosage and ratios is a matter of routine determination, as described in the example section below, and within the skill of a practitioner of ordinary skill, in light of the disclosure provided herein.

In treating a prostate cancer patient using the compositions and methods of the invention, the attending physician may administer lower doses of the cytokine-expressing tumor cell vaccine and observe the patient's response. Larger doses of the cytokine-expressing tumor cell vaccine may be administered until the an improved therapeutic outcome is evident.

Cytokine-producing cells of the invention are processed to remove most additional components used in preparing the cells. In particular, fetal calf serum, bovine serum components, or other biological supplements in the culture medium are removed. In one embodiment, the cells are washed, such as by repeated gentle centrifugation, into a suitable pharmacologically compatible excipient. Compatible excipients include various cell culture media, isotonic saline, with or without a physiologically compatible buffer like phosphate or Hepes and nutrients such as dextrose, physiologically compatible ions, or amino acids, particularly those devoid of other immunogenic components. Carrying reagents, such as albumin and blood plasma fractions and nonactive thickening agents, may also be used.

Autologous

The use of autologous genetically modified GM-CSF expressing cells provides advantages since each patient's tumor expresses a unique set of tumor antigens that can differ from those found on histologically-similar, MHC-matched tumor cells from another patient. See, e.g., Kawakami et al., J. Immunol., 148, 638-643 (1992); Darrow et al., J. Immunol., 142, 3329-3335 (1989); and Hom et al., J. Immunother., 10, 153-164 (1991). In contrast, MHC-matched tumor cells provide the advantage that the patient need not be taken to surgery to obtain a sample of their tumor for genetically modified tumor cell production.

In one preferred aspect, the present invention comprises a method of treating prostate cancer by carrying out the steps of: (a) obtaining tumor cells from a mammalian subject harboring a prostate tumor; (b) genetically modifying the tumor cells to render them capable of producing an increased level of GM-CSF relative to unmodified tumor cells; (c) rendering the modified tumor cells proliferation incompetent; and (d) readministering the genetically modified tumor cells to the mammalian subject from which the tumor cells were obtained or to a mammal with the same MHC type as the mammal from which the tumor cells were obtained. The administered tumor cells are autologous and MHC-matched to the host. Preferably, the composition is administered intradermally, subcutaneously or intratumorally to the mammalian subject.

In some cases, a single autologous tumor cell may express GM-CSF alone or GM-CSF plus one or more additional transgenes. In other cases, GM-CSF and the one or more additional transgenes may be expressed by different autologous tumor cells. In one aspect of the invention, an autologous tumor cell is modified by introduction of a vector comprising a nucleic acid sequence encoding GM-CSF, operatively linked to a promoter and expression/control sequences necessary for expression thereof. In another aspect, the same autologous tumor cell or a second autologous tumor cell is modified by introduction of a vector comprising a nucleic acid sequence encoding at least one additional transgene operatively linked to a promoter and expression/control sequences necessary for expression thereof. The nucleic acid sequence encoding the one or more transgenes are introduced into the same or a different autologous tumor cell using the same or a different vector. The nucleic acid sequence encoding the transgene(s) may or may not further comprise a selectable marker sequence operatively linked to a promoter. Desirably, the autologous tumor cell expresses high levels of GM-CSF.

Allogeneic

Researchers have sought alternatives to autologous and MHC-matched cells as tumor vaccines, as reviewed by Jaffee et al., Seminars in Oncology, 22, 81-91 (1995). Early tumor vaccine strategies were based on the understanding that the vaccinating cells function as the antigen presenting cells (APCs) that present tumor antigens on their MHC class I and II molecules, and directly activate the T cell arm of the immune system. The results of Huang et al. (Science, 264, 961-965, 1994), indicate that professional APCs of the host rather than the vaccinating cells prime the T cell arm of the immune system by secreting cytokine(s) such as GM-CSF such that bone marrow-derived APCs are recruited to the region of the tumor. The bone marrow-derived APCs take up the whole cellular protein of the tumor for processing, and then present the antigenic peptide(s) on their MHC class I and II molecules, thereby priming both the CD4+ and the CD8+ T cell arms of the immune system, resulting in a systemic tumor-specific anti-tumor immune response. Without being bound by theory, these results suggest that it may not be necessary or optimal to use autologous or MHC-matched cells in order to elicit an anti-cancer immune response and that the transfer of allogeneic MHC genes (from a genetically dissimilar individual of the same species) can enhance tumor immunogenicity. More specifically, in certain cases, the rejection of tumors expressing allogeneic MHC class I molecules has resulted in enhanced systemic immune responses against subsequent challenge with the unmodified parental tumor. See, e.g., Jaffee et al., supra, and Huang et al., supra.

As described herein, a “tumor cell line” comprises cells that were initially derived from a tumor. Such cells typically are transformed (i.e., exhibit indefinite growth in culture). In one preferred aspect, the invention provides a method for treating prostate cancer by carrying out the steps of: (a) obtaining a tumor cell line; (b) genetically modifying the tumor cell line to render the cells capable of producing an increased level of a cytokine, e.g., GM-CSF, relative to the unmodified tumor cell line; (c) rendering the modified tumor cell line proliferation incompetent; and (d) administering the tumor cell line to a mammalian subject (host) having at least one tumor that is of the same type of tumor as that from which the tumor cell line was obtained. The administered tumor cell line is allogeneic and is not MHC-matched to the host. Such allogeneic lines provide the advantage that they can be prepared in advance, characterized, aliquoted in vials containing known numbers of transgene (e.g., GM-CSF) expressing cells and stored (i.e. frozen) such that well characterized cells are available for administration to the patient. Methods for the production of genetically modified allogeneic cells are described for example in WO 00/72686, expressly incorporated by reference herein.

In one approach to preparing genetically modified GM-CSF expressing allogeneic cells, a nucleic acid sequence (transgene) encoding GM-CSF alone or in combination with the nucleic acid coding sequence for one or more additional transgenes is introduced into a cell line that is an allogeneic tumor cell line (i.e., derived from an individual other than the individual being treated). In another approach, a nucleic acid sequence (transgene) encoding GM-CSF alone or in combination with the nucleic acid coding sequence for one or more additional transgenes is introduced into separate allogeneic tumor cell lines. In yet another approach two or more different genetically modified allogeneic GM-CSF expressing cell lines (e.g. LNCAP and PC-3) are administered in combination, typically at a ratio of 1:1. In general, the cell or population of cells is from a tumor cell line of the same type as the tumor or cancer being treated, e.g. prostate cancer. The nucleic acid sequence encoding the transgene(s) may be introduced into the same or a different allogeneic tumor cell using the same or a different vector. The nucleic acid sequence encoding the transgene(s) may or may not further comprise a selectable marker sequence operatively linked to a promoter. Desirably, the allogeneic cell line expresses high levels of GM-CSF.

In another aspect of the invention, one or more genetically modified GM-CSF expressing allogeneic cell lines are exposed to an antigen, such that the patient's immune response to the antigen is increased in the presence of GM-CSF, e.g., an allogeneic or bystander cell that has been genetically modified to express GM-CSF. Such exposure may take place ex vivo or in vivo. In one preferred embodiment, the antigen is an approximately 278 kD antigen as determined by SDS-PAGE, identified as beta filamin. Beta filamin is provided by (on) cells that are administered to the subject or may be provided by cells native to the patient. In such cases, the composition is rendered proliferation-incompetent, typically by irradiation, wherein the allogeneic cells are plated in a tissue culture plate and irradiated at room temperature using a Cs source, as further described herein. An allogeneic cellular vaccine composition of the invention may comprise allogeneic cells plus other cells, i.e. a different type of allogeneic cell, an autologous cell, or a bystander cell that may or may not be genetically modified. If genetically modified, the different type of allogeneic cell, autologous cell, or bystander cell may express GM-CSF or another transgene. The ratio of allogeneic cells to other cells in a given administration will vary dependent upon the combination.

Any suitable route of administration can be used to introduce an allogeneic cell line composition into the patient, preferably, the composition is administered intradermally, subcutaneously or intratumorally.

The use of allogeneic cell lines in practicing the present invention provides the therapeutic advantage that administration of a genetically modified GM-CSF expressing cell line to a patient with cancer, together with an autologous cancer antigen, paracrine production of GM-CSF results in an effective immune response to a tumor. This obviates the need to culture and transduce autologous tumor cells for each patient.

Bystander

In one further aspect, the present invention provides a universal immunomodulatory genetically modified transgene-expressing bystander cell that expresses at least one transgene. The same universal bystander cell line may express more than one transgene or individual transgenes may be expressed by different universal bystander cell lines. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect of the invention, a universal bystander cell line is modified by introduction of a vector wherein the vector comprises a nucleic acid sequence encoding a transgene, e.g., a cytokine such as GM-CSF, operably linked to a promoter and expression control sequences necessary for expression thereof. In another aspect, the same universal bystander cell line or a second a universal bystander cell line is modified by introduction of a vector comprising a nucleic acid sequence encoding at least one additional transgene operatively linked to a promoter and expression control sequences necessary for expression thereof. The nucleic acid sequence encoding the transgene(s) may be introduced into the same or a different universal bystander cell line using the same or a different vector. The nucleic acid sequence encoding the transgene(s) may or may not further comprise a selectable marker sequence operatively linked to a promoter. Any combination of transgene(s) that stimulate an anti-tumor immune response finds utility in the practice of the present invention. The universal bystander cell line preferably grows in defined, i.e., serum-free medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of the generation of human bystander cell lines is described for example in U.S. Pat. No. 6,464,973, expressly incorporated by reference herein.

Desirably, the universal bystander cell line expresses high levels of the transgene, e.g. a cytokine such as GM-CSF.

In practicing the invention, the one or more universal bystander cell lines are incubated with an autologous cancer antigen, e.g., provided by an autologous tumor cell (which together comprise a universal bystander cell line composition), then the universal bystander cell line composition is administered to the patient. Any suitable route of administration can be used to introduce a universal bystander cell line composition into the patient. Preferably, the composition is administered intradernally, subcutaneously or intratumorally.

Typically, the autologous cancer antigen is provided by a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed above.

The ratio of bystander cells to autologous cancer cells in a given administration will vary dependent upon the combination. With respect to GM-CSF-producing bystander cells, the ratio of bystander cells to autologous cancer cells in a given administration should be such that a therapeutically effective level of GM-CSF is produced. In addition to the GM-CSF threshold, the ratio of bystander cells to autologous cancer cells should not be greater than 1:1. Appropriate ratios of bystander cells to tumor cells or tumor antigens can be determined using routine methods known in the art.

The use of bystander cell lines in practicing the present invention provides the therapeutic advantage that, through administration of a cytokine-expressing bystander cell line and at least one additional cancer therapeutic agent (expressed by the same or a different cell) to a patient with cancer, together with an autologous cancer antigen, paracrine production of an immunomodulatory cytokine, results in an effective immune response to a tumor. This obviates the need to culture and transduce autologous tumor cells for each patient.

Typically a minimum dose of about 3500 Rads is sufficient to inactivate a cell and render it proliferation-incompetent, although doses up to about 30,000 Rads are acceptable. In some embodiment, the cells are irradiated at a dose of from about 50 to about 200 rads/min or from about 120 to about 140 rads/min prior to administration to the mammal. Typically, when using irradiation, the levels required are 2,500 rads, 5,000 rads, 10,000 rads, 15,000 rads or 20,000 rads. In one embodiment, a dose of about 10,000 Rads is used to inactivate a cell and render it proliferation-incompetent. It is understood that irradiation is but one way to render cells proliferation-incompetent, and that other methods of inactivation which result in cells incapable of multiple rounds of cell division but that retain the ability to express transgenes (e.g. cytokines) are included in the present invention (e.g., treatment with mitomycin C, cycloheximide, and conceptually analogous agents, or incorporation of a suicide gene by the cell).

Cytokines

A “cytokine” or grammatical equivalent, includes, without limitation, those hormones that act locally and do not circulate in the blood, and which, when used in accordance with the present invention, will result in an alteration of an individual's immune response. Also included in the definition of cytokine are adhesion or accessory molecules which result in an alteration of an individual's immune response. Thus, examples of cytokines include, but are not limited to, IL-1 (a or P), IL-2, IL-3, IL-4, IL-5, IL-6, IL-7, IL-8, IL-9, IL-10, IL-11, IL-12, GM-CSF, M-CSF, G-CSF, LIF, LT, TGF-P, y-IFN, a-EFN, P-IFN, TNF-a, BCGF, CD2, or ICAM. Descriptions of the aforementioned cytokines as well as other applicable immunomodulatory agents may be found in “Cytokines and Cytokine Receptors,” A. S. Hamblin, D. Male (ed.), Oxford University Press, New York, N.Y. (1993)), or the “Guidebook to Cytokines and Their Receptors,” N. A. Nicola (ed.), Oxford University Press, New York, N.Y. (1995)). Where therapeutic use in humans is contemplated, the cytokines will preferably be substantially similar to the human form of the protein or will have been derived from human sequences (i.e., of human origin). In one preferred embodiment, the transgene is a cytokine, such as GM-CSF.

Additionally, cytokines of other mammals with substantial structural homology and/or amino acid sequence identity to the human forms of a given cytokine, will be useful in the invention when demonstrated to exhibit similar activity on the human immune system. Similarly, proteins that are substantially analogous to any particular cytokine, but have conservative changes of protein sequence, will also find use in the present invention. Thus, conservative substitutions in protein sequence may be possible without disturbing the functional abilities of the protein molecule, and thus proteins can be made that function as cytokines in the present invention but have amino acid sequences that differ slightly from currently known sequences. Such conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, it also activates the antigen processing and presenting function of dendritic cells, which are the major antigen presenting cells (APC) of the immune system. Results from animal model experiments have convincingly shown that GM-CSF producing cells (i.e. GVAX®) are able to induce an immune response against parental, non-transduced cells.

GM-CSF augments the antigen presentation capability of the subclass of dendritic cells (DC) capable of stimulating robust anti-tumor responses (Gasson et al. Blood 1991 Mar. 15;77(6):1131-45; Mach et al. Cancer Res. 2000 Jun. 15;60(12):3239-46; reviewed in Mach and Dranoff, Curr Opin Immunol. 2000 October; 12(5):571-5). See, e.g., Boon and Old, Curr Opin Immunol. 1997 Oct. 1; 9(5):681-3). Presentation of tumor antigen epitopes to T cells in the draining lymph nodes is expected to result in systemic immune responses to tumor metastases. Also, irradiated tumor cells expressing GM-CSF have been shown to function as potent vaccines against tumor challenge (as further described in the section below, entitled “GVAX®”). Localized high concentrations of certain cytokines, delivered by genetically modified cells, have been found to lead to tumor regression (Abe et al., J. Canc. Res. Clin. Oncol. 121: 587-592 (1995); Gansbacher et al., Cancer Res. 50: 7820-7825 (1990); Formi et al., Cancer and Met. Reviews 7: 289-309 (1988). PCT publication WO200072686 describes tumor cells expressing various cytokines.

In one embodiment of the invention, the cellular vaccine comprises a GM-CSF coding sequence operatively linked to regulatory elements for expression in the cells of the vaccine. The GM-CSF coding sequence may code for a murine or human GM-CSF and may be in the form of genomic DNA (SEQ ID NO:1) or cDNA (SEQ ID NO:2). In the case of cDNA, the coding sequence for GM-CSF does not contain intronic sequences to be spliced out prior to translation. In contrast, for genomic GM-CSF, the coding sequence contains at least one native GM-CSF intron that is spliced out prior to translation. In one embodiment, the GM-CSF coding sequence codes for SEQ ID NO:3. Other examples of GM-CSF coding sequences are found in Genbank accession numbers: AF373868, AC034228, AC034216, M 10663 and NM000758.

A GM-CSF coding sequence according to the present invention may be a full-length complement that hybridizes to the sequence shown in SEQ ID NO:1 or SEQ ID NO:2 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

It follows that, according to the present invention the coding sequence for a cytokine such as GM-CSF, has at least 80, 85, 87, 89, 90, 91, 92, 93, 94, 95, 96, 97, 98, 99% or more % identity over its entire length to a native GM-CSF coding sequence. For example, a GM-CSF coding sequence according to the present invention has at least 80, 85, 87, 89, 90, 91, 92, 93, 94, 95, 96, 97, 98, 99% or more sequence identity to a sequence presented as SEQ ID NO:1 or SEQ ID NO:2, when compared and aligned for maximum correspondence, as measured a sequence comparison algorithm (as described above) or by visual inspection. In one embodiment, the given % sequence identity exists over a region of the sequences that is at least about 50 nucleotides in length. In another embodiment, the given % sequence identity exists over a region of at least about 100 nucleotides in length. In another embodiment, the given % sequence identity exists over a region of at least about 200 nucleotides in length. In another embodiment, the given % sequence identity exists over the entire length of the sequence.

In addition according to the present invention, the amino acid sequence for a cytokine such as GM-CSF has at least 80, 85, 87, 89, 90, 91, 92, 93, 94, 95, 96, 97, 98, 99% or more sequence identity to the sequence presented as SEQ ID NO:3, when compared and aligned for maximum correspondence.

In one embodiment, cells are engineered (genetically modified) to enhance expression of an antigen associated with an immune response to prostate cancer (e.g., beta filamin) and are either further engineered to express one or more proteins that enhance the immune response to prostate cancer, e.g., a cytokine such as GM-CSF or are administered in combination with different cells which are either further engineered to express one or more proteins that enhance the immune response to prostate cancer, e.g., a cytokine such as GM-CSF.

Beta Filamin

One embodiment of the invention is a method of treating prostate cancer in manner that results in an enhanced immune response to an antigen such as beta filamin, wherein the enhanced immune response is associated with an improved therapeutic outcome for the subject, for example, a reduction in the level of PSA in the patient's serum, a decrease in cancer-associated pain or improvement in the condition of the patient according to any clinically acceptable criteria, including but not limited to a decrease in metastases, an increase in life expectancy or an improvement in quality of life. The beta filamin may be expressed endogenously by cells native to the patient or may be exogenously provided to the subject (patient).

Mammals have three filamin genes, Filamin-A, Filamin-B (beta-filamin; Filamin-3) and Filamin-C. Human filamins are 280-kDa proteins containing an N-terminal actin-binding domain followed by 24 characteristic repeats. They also interact with a number of other cellular proteins. The filamins usually are found as approximately 560-kDA homodimers or heterodimers formed with other filamins. Beta-filamin is also known as ABP-278/276 (Xu et al. 1998 Blood 92:1268-1276). See, e.g., Takafuta et al. 1998 J Biol Chem 273:17531-17538; Flier et al., J. Cell Biol., 156(2)361-376, 2002. The 2602 amino acid beta filamin protein sequence may be found at GenBank Accession Nos. NP_{—}001448. The expression patterns of Filamin B and Filamin-A is described for example in Sheen et al., Human Mol. Gen. 11(23) 2845-2854, 2002. Leedman et al., Proc Natl Acad Sci USA. 90(13):5994-8, 1993 describe the cloning of a protein related to actin binding protein, later designated beta filamin.

In one embodiment according to the present invention, the coding sequence for an antigen associated with an immune response to prostate cancer (e.g., beta filamin) has a full-length complement that hybridizes to the sequence shown in SEQ ID NO:4 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

It follows that, according to the present invention the coding sequence for an antigen associated with an immune response to prostate cancer (e.g., beta filamin), has at least 80, 85, 87, 89, 90, 91, 92, 93, 94, 95, 96, 97, 98, 99% or more % identity over its entire length to the native coding sequence. For example, a beta filamin coding sequence according to the present invention has at least 80, 85, 87, 89, 90, 91, 92, 93, 94, 95, 96, 97, 98, 99% or more sequence identity to the sequence presented as SEQ ID NO:4, when compared and aligned for maximum correspondence, as measured a sequence comparison algorithm (as described above) or by visual inspection. In one embodiment, the given % sequence identity exists over a region of the sequences that is at least about 50 nucleotides in length. In another embodiment, the given % sequence identity exists over a region of at least about 100 nucleotides in length. In another embodiment, the given % sequence identity exists over a region of at least about 200 nucleotides in length. In another embodiment, the given % sequence identity exists over the entire length of the sequence.

In addition according to the present invention, the amino acid sequence for an antigen associated with an immune response to prostate cancer (e.g., beta filamin) has at least 80, 85, 87, 89, 90, 91, 92, 93, 94, 95, 96, 97, 98, 99% or more sequence identity to the native beta-filamin sequence presented as SEQ ID NO:5, when compared and aligned for maximum correspondence.

In practicing the invention the immune response to beta filamin is enhanced following administration of a cytokine-expressing cellular vaccine, e.g., a GM-CSF-expressing tumor cell (or GVAX®) to a patient.

In one embodiment of the invention a population of cells that expresses beta-filamin is administered to the patient as part of a cellular vaccine. The beta-filamin expressing cell may be the same as or different from the cell that expresses a cytokine such as GM-CSF. In another embodiment of the invention, the cellular vaccine is comprised of purified beta-filamin protein or immunogenic fragments thereof. The cellular vaccine may further comprise an immune enhancing agent (e.g. a cytokine such as GM-CSF, adjuvant or the like). In practicing the invention, the full-length beta filamin protein may be used as an antigen. Those of skill in the art, however, will appreciate that a detected immune response to beta filamin may also be evident following exposure of the subject to a fragment of beta filamin or upon administration of a cellular vaccine, such as GVAX®, in the absence of exogenously provided beta filamin. Beta filamin fragments may comprise linear segments of the full-length amino acid sequence or alternative splicing variants, deletion mutants or other mutants. To be useful with the present invention, beta filamin fragments should be immunogenic fragments, i.e. capable of eliciting an immune response.

Cells can be enhanced for beta-filamin expression by various methods known to those skilled in the art. For example, cells may be transduced with a vector which encodes beta-filamin, operatively linked to the beta-filamin coding sequence. Suitable promoters are known and available to those skilled in the art. A vector useful for transducing the cells can be any vector that is effective to result in the enhanced expression of beta-filamin. In one embodiment the vector is a viral vector, e.g., a retroviral vector such as a lentiviral vector, an adenoviral vector or an adeno-associated viral vector. A vector may also be used to transduce the cells with a coding region for a protein that enhances the immune response to cancer in the subject, e.g., a cytokine such as GM-CSF. This coding region and the beta-filamin coding region can be located on one vector or on separate vectors and introduced into the same or different cells. If on separate vectors, the separate vectors may be of the same origin (e.g. retroviral) or of different origins. In one embodiment, the cell is first transduced with a vector coding for beta-filamin and then transduced with a vector coding for GM-CSF. In another embodiment, the cell is first transduced with a vector coding for at least one protein that enhances an immune response to prostate cancer and then transduced with a vector coding for beta-filamin.

In one embodiment of the invention, the beta-filamin coding sequence in the vector is the native sequence (GenBank NM_{—}001457; SEQ ID NO: 4) or a “recoded” sequence. A gene that is “recoded” refers to a coding sequence that is altered in such a manner that the polypeptide encoded by a nucleic acid remains the same as in the unaltered sequence but the nucleic acid sequence encoding the polypeptide is changed. It is well known in the art that due to degeneracy of the genetic code, there exist multiple DNA and RNA codons which can encode the same amino acid translation product. Furthermore, it is also known that different organisms have different preferences for utilization of particular codons to synthesize an amino acid. Therefore, in one embodiment of the invention the vector contains a beta-filamin coding sequence that has been recoded with preferred codons for humans. In one embodiment, the beta-filamin coding sequence codes for an alternatively spliced form of beta-filamin. Alternatively spliced forms of beta-filamin are described in GenBank Accession numbers AF353666 and AF353667 and van der Flier et al.

Another embodiment of the invention is a method of increasing an immune response to a tumor cell and/or beta-filamin comprising: administering genetically modified cytokine-expressing cells of the invention to a prostate cancer patient wherein an improved therapeutic outcome results. Another embodiment of the invention is a method of increasing an immune response to a tumor cell and/or beta-filamin comprising: administering genetically modified cytokine-expressing cells that exhibit enhanced expression of a beta-filamin to a prostate cancer patient wherein after said administration, the patient's immune response to prostate cancer is increased. Yet another embodiment of the invention is a method of increasing an immune response to a tumor cell and beta-filamin comprising: administering genetically modified cells that exhibit enhanced expression of a cytokine (e.g., GM-CSF) to a prostate cancer patient, wherein after administration, the mammal's immune response to beta-filamin is increased. In one embodiment, the increased immune response is humoral. In yet a another embodiment, the increased immune response is cellular. In still a further embodiment, the increased immune response is both cellular and humoral. In a preferred aspect of the invention, after administration of genetically modified cytokine-producing cells, the growth of the prostate cancer cells is inhibited.

Assays for determining if the cells express detectable levels of beta-filamin and/or if the immune response to beta filamin has changed following administration of a cytokine-expressing cell vaccine include, but are not limited to, ELISA, Western blot, Immunofluorescence assay (IFA), FACS or Electrochemiluminescence (ECL).

Genetic Modification of Cells for Use as Cancer Vaccines

The methods and compositions of the invention are exemplified in detail herein by particular vector systems, however, one of skill in the art will readily appreciate that the same methods and compositions find utility in the treatment of prostate cancer independent of the gene delivery system.

The present invention contemplates the use of any vector for introduction of transgenes such as GM-CSF or beta-filamin into mammalian cells. Exemplary vectors include but are not limited to, viral and non-viral vectors, such as retroviruses (including lentiviruses), adenovirus (Ad) vectors including replication competent, replication deficient and gutless forms thereof, adeno-associated virus (AAV) vectors, simian virus 40 (SV-40) vectors, bovine papilloma virus vectors, Epstein-Barr virus vectors, herpes virus vectors, vaccinia virus vectors, Moloney murine leukemia virus vectors, Harvey murine sarcoma virus vectors, murine mammary tumor virus vectors, Rous sarcoma virus vectors and nonviral plasmid vectors. In one preferred approach, the vector is a viral vector. Viruses can efficiently transduce cells and introduce their own DNA into a host cell. In generating recombinant viral vectors, non-essential genes are replaced with a gene or coding sequence for a heterologous (or non-native) protein.

In constructing viral vectors, non-essential genes are replaced with one or more genes encoding one or more therapeutic compounds or factors. Typically, the vector comprises an origin of replication and the vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern et al., J., J Mol Appl Genet. 1982;1(4):327-41 (1982)), mycophenolic acid (Mulligan et al., Science 209:1422-7 (1980)), puromycin, zeomycin, hygromycin (Sugden et al., Mol Cell Biol. 5(2):410-3 (1985)) or G418.

Adenovirus gene therapy vectors are known to exhibit strong expression in vitro, excellent titer, and the ability to transduce dividing and non-dividing cells in vivo (Hitt et al., Adv in Virus Res 55:479-505 (2000)). When used in vivo these vectors lead to strong but transient gene expression due to immune responses elicited to the vector backbone. The recombinant Ad vectors for use in the instant invention comprise: (1) a packaging site enabling the vector to be incorporated into replication-defective Ad virions; and (2) a therapeutic compound coding sequence. Other elements necessary or helpful for incorporation into infectious virions, include the 5′ and 3′ Ad ITRs, the E2 and E3 genes, etc.

Replication-defective Ad virions encapsulating the recombinant Ad vectors of the instant invention are made by standard techniques known in the art using Ad packaging cells and packaging technology. Examples of these methods may be found, for example, in U.S. Pat. No. 5,872,005, incorporated herein by reference in its entirety. A therapeutic compound-encoding gene is commonly inserted into adenovirus in the deleted E1A, E1B or E3 region of the virus genome. Preferred adenoviral vectors for use in practicing the invention do not express one or more wild-type Ad gene products, e.g., E1a, E1b, E2, E3, E4. Preferred embodiments are virions that are typically used together with packaging cell lines that complement the functions of E1, E2A, E4 and optionally the E3 gene regions. See, e.g. U.S. Pat. Nos. 5,872,005, 5,994,106, 6,133,028 and 6,127,175, expressly incorporated by reference herein in their entirety. Adenovirus vectors are purified and formulated using standard techniques known in the art.

Recombinant AAV vectors are characterized in that they are capable of directing the expression and the production of the selected transgenic products in targeted cells. Thus, the recombinant vectors comprise at least all of the sequences of AAV essential for encapsidation and the physical structures for infection of target cells.

Recombinant AAV (rAAV) virions for use in practicing the present invention may be produced using standard methodology, known to those of skill in the art and are constructed such that they include, as operatively linked components in the direction of transcription, control sequences including transcriptional initiation and termination sequences, and the coding sequence for a therapeutic compound or biologically active fragment thereof. These components are bounded on the 5′ and 3′ end by functional AAV ITR sequences. By “functional AAV ITR sequences” is meant that the ITR sequences function as intended for the rescue, replication and packaging of the AAV virion. Hence, AAV ITRs for use in the vectors of the invention need not have a wild-type nucleotide sequence, and may be altered by the insertion, deletion or substitution of nucleotides or the AAV ITRs may be derived from any of several AAV serotypes. An AAV vector is a vector derived from an adeno-associated virus serotype, including without limitation, AAV-1, AAV-2, AAV-3, AAV-4, AAV-5, AAV-6, AAV-7, AAV-8, etc. Preferred AAV vectors have the wild type REP and CAP genes deleted in whole or part, but retain functional flanking ITR sequences. Table 1 illustrates exemplary AAV serotypes for use in gene transfer.

TABLE 1
AAV Serotypes For Use In Gene Transfer.

| | | Genome Size | Homology vs | Immunity in |
|---|---|---|---|---|
| Serotype | Origin | (bp) | AAV2 | Human Population |
| AAV-1 | Human Specimen | 4718 | NT: 80% | NAB: 20% |
| | | | AA: 83% | |
| AAV-2 | Human Genital Abortion | 4681 | NT: 100% | NAB: 27-53% |
| | Tissue Amnion Fluid | | AA: 100% | |
| AAV-3 | Human Adenovirus | 4726 | NT: 82% | cross reactivity with |
| | Specimen | | AA: 88% | AAV2 NAB |
| AAV-4 | African Green Monkey | 4774 | NT: 66% | Unknown |
| | | | AA: 60% | |
| AAV-5 | Human Genital Lesion | 4625 | NT: 65% | ELISA: 45% NAB: 0% |
| | | | AA: 56% | |
| AAV-6 | Laboratory Isolate | 4683 | NT: 80% | 20% |
| | | | AA: 83% | |
| AAV-7 | Isolated from Heart DNA | 4721 | NT: 78% | NAB: <1:20 (˜5%) |
| | of Rhesus Monkey | | AA: 82% | |
| AAV-8 | Isolated from Heart DNA | 4393 | NT: 79% | NAB: <1:20 (˜5%) |
| | of Rhesus Monkey | | AA: 83% | |

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large REP proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety, and include those techniques within the knowledge of those of skill in the art.

A large number of serotypes of AAV are currently known, however, new serotypes and variants of existing serotypes are still being identified today and are considered within the scope of the present invention. See Gao et al (2002), PNAS 99(18):11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue, such as the brain. The use of different AAV serotypes may facilitate targeting of malignant tissue. AAV serotypes including 1, 2, 4, 5 and 6 have been shown to transduce brain tissue. See, e.g., Davidson et al (2000), PNAS 97(7)3428-32; Passini et al (2003), J. Virol 77(12):7034-40). Particular AAV serotypes may more efficiently target and/or replicate in target tissue or cells. A single self-complementary AAV vector can be used in practicing the invention in order to increase transduction efficiency and result in faster onset of transgene expression (McCarty et al., Gene Ther. 2001 August; 8(16): 1248-54).

In practicing the invention, host cells for producing rAAV virions include mammalian cells, insect cells, microorganisms and yeast. Host cells can also be packaging cells in which the AAV REP and CAP genes are stably maintained in the host cell or alternatively host cells can be producer cells in which the AAV vector genome is stably maintained. Exemplary packaging and producer cells are derived from 293, A549 or HeLa cells. AAV vectors are purified and formulated using standard techniques known in the art.

Retroviral vectors are a common tool for gene delivery (Miller, 1992, Nature 357: 455-460). Retroviral vectors and more particularly lentiviral vectors may be used in practicing the present invention. Retroviral vectors have been tested and found to be suitable delivery vehicles for the stable introduction of a variety of genes of interest into the genomic DNA of a broad range of target cells. The ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) may also find utility in practicing the present invention. The ability to direct the delivery of retroviral vectors encoding a transgene to a specific type of target cells is highly desirable for gene therapy applications.

The present invention provides retroviral vectors which include e.g., retroviral transfer vectors comprising one or more transgene sequences and retroviral packaging vectors comprising one or more packaging elements. In particular, the present invention provides pseudotyped retroviral vectors encoding a heterologous or functionally modified envelope protein for producing pseudotyped retrovirus.

The core sequence of the retroviral vectors of the present invention may be readily derived from a wide variety of retroviruses, including for example, B, C, and D type retroviruses as well as spumaviruses and lentiviruses (see RNA Tumor Viruses, Second Edition, Cold Spring Harbor Laboratory, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Particularly preferred Murine Leukemia Viruses include 4070A and 1504A (Hartley and Rowe, J. Virol. 19:19-25, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (“ATCC”; Rockville, Md.), or isolated from known sources using commonly available techniques.

Preferably, a retroviral vector sequence of the present invention is derived from a lentivirus. A preferred lentivirus is a human immunodeficiency virus, e.g., type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. Other lentivirus vectors include, a sheep Visnalmaedi virus, a feline immunodeficiency virus (FIV), a bovine lentivirus, simian immunodeficiency virus (SIV), an equine infectious anemia virus (EIAV), and a caprine arthritis-encephalitis virus (CAEV).

The various genera and strains of retroviruses suitable for use in the compositions and methods are well known in the art (see, e.g., Fields Virology, Third Edition, edited by B.N. Fields et al., Lippincott-Raven Publishers (1996), see e.g., Chapter 58, Retroviridae: The Viruses and Their Replication, Classification, pages 1768-1771, including Table 1, incorporated herein by reference).

The present invention provides retroviral packaging systems for generating producer cells and producer cell lines that produce retroviruses, and methods of making such packaging systems. Accordingly, the present invention also provides producer cells and cell lines generated by introducing a retroviral transfer vector into such packaging systems (e.g., by transfection or infection), and methods of making such packaging cells and cell lines.

The retroviral packaging systems for use in practicing the present invention comprise at least two packaging vectors: a first packaging vector which comprises a first nucleotide sequence comprising a gag, a pol, or gag and pol genes; and a second packaging vector which comprises a second nucleotide sequence comprising a heterologous or functionally modified envelope gene. In a preferred embodiment, the retroviral elements are derived from a lentivirus, such as HIV. Preferably, the vectors lack a functional tat gene and/or functional accessory genes (vif, vpr, vpu, vpx, nef). In another preferred embodiment, the system further comprises a third packaging vector that comprises a nucleotide sequence comprising a rev gene. The packaging system can be provided in the form of a packaging cell that contains the first, second, and, optionally, third nucleotide sequences.

The invention is applicable to a variety of retroviral systems, and those skilled in the art will appreciate the common elements shared across differing groups of retroviruses. The description herein uses lentiviral systems as a representative example. However, all retroviruses share the features of enveloped virions with surface projections and containing one molecule of linear, positive-sense single stranded RNA, a genome consisting of a dimer, and the common proteins gag, pol and env.

Lentiviruses share several structural virion proteins in common, including the envelope glycoproteins SU (gp120) and TM (gp41), which are encoded by the env gene; CA (p24), MA (p17) and NC (p7-11), which are encoded by the gag gene; and RT, PR and IN encoded by the pol gene. HIV-1 and HIV-2 contain accessory and other proteins involved in regulation of synthesis and processing virus RNA and other replicative functions. The accessory proteins, encoded by the vif, vpr, vpu/vpx, and nef genes, can be omitted (or inactivated) from the recombinant system. In addition, tat and rev can be omitted or inactivated, e.g., by mutation or deletion.

First generation lentiviral vector packaging systems provide separate packaging constructs for gag/pol and env, and typically employ a heterologous or functionally modified envelope protein for safety reasons. In second generation lentiviral vector systems, the accessory genes, vif, vpr, vpu and nef, are deleted or inactivated. Third generation lentiviral vector systems are those from which the tat gene has been deleted or otherwise inactivated (e.g., via mutation).

Compensation for the regulation of transcription normally provided by tat can be provided by the use of a strong constitutive promoter, such as the human cytomegalovirus immediate early (HCMV-IE) enhancer/promoter. Other promoters/enhancers can be selected based on strength of constitutive promoter activity, specificity for target tissue (e.g., liver-specific promoter), or other factors relating to desired control over expression, as is understood in the art. For example, in some embodiments, it is desirable to employ an inducible promoter such as tet to achieve controlled expression. The gene encoding rev is preferably provided on a separate expression construct, such that a typical third generation lentiviral vector system will involve four plasmids: one each for gagpol, rev, envelope and the transfer vector. Regardless of the generation of packaging system employed, gag and pol can be provided on a single construct or on separate constructs.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, DHFR, Gln synthetase or ADA, followed by selection in the presence of the appropriate drug and isolation of clones. A selectable marker gene can be linked physically to genes encoding by the packaging vector.

Stable cell lines, wherein the packaging functions are configured to be expressed by a suitable packaging cell, are known. For example, see U.S. Pat. No. 5,686,279; and Oryet al., Proc. Natl. Acad. Sci. (1996) 93:11400-11406, which describe packaging cells. Further description of stable cell line production can be found in Dull et al., 1998, J. Virology 72(11):8463-8471; and in Zufferey et al., 1998, J. Virology 72(12):9873-9880; Zufferey et al., 1997, Nature Biotechnology 15:871-875, teach a lentiviral packaging plasmid wherein sequences 3′ of pol including the HIV-1 envelope gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

The packaging vectors of interest may contain additional changes to the packaging functions to enhance lentiviral protein expression and to enhance safety. For example, all of the HIV sequences upstream of gag can be removed. Also, sequences downstream of envelope can be removed. Moreover, steps can be taken to modify the vector to enhance the splicing and translation of the RNA. Optionally, a conditional packaging system is used, such as that described by Dull et al., 1998, J. Virology 72(11):8463-8471. Also preferred is the use of a self-inactivating vector (SIN), which improves the biosafety of the vector by deletion of the HIV-1 long terminal repeat (LTR) as described, for example, by Zufferey et al., 1998, J. Virology 72(12):9873-9880. Inducible vectors can also be used, such as through a tet-inducible LTR.

Regulatory Elements

The gene therapy vectors of the invention typically include heterologous control sequences, which include, but are not limited to constitutive promoters, inducible promoters, tumor selective promoters and enhancers, including but not limited to the E2F promoter and the telomerase (hTERT) promoter; the cytomegalovirus enhancer/chicken beta-actin/Rabbit β-globin promoter (CAG promoter; Niwa H. et al. 1991. Gene 108(2):193-9); the elongation factor 1-alpha promoter (EF1-alpha) promoter (Kim D W et al. 1990. Gene. 91(2):217-23 and Guo Z S et al. 1996. Gene Ther. 3(9):802-10); a glial specific promoter (e.g. glial fibrary acid protein promoter) and a neuron specific promoter (e.g. neuron specific enolase promoter or synapsin promoter).

In some cases constitutive promoters, such as the cytomegalovirus (CMV) immediate early promoter, the RSV LTR, the MoMLV LTR, the CAG promoter, the phosphoglycerate kinase-1 promoter (PGK) or the SV-40 promoter may be employed. The gene therapy vectors of the invention may also include enhancers and coding sequences for signal peptides. The vector constructs may or may not include an intron. Thus it will be appreciated that gene therapy vectors of the invention may include any of a number of transgenes, combinations of transgenes and transgene/regulatory element combinations.

Methods and Compositions of the Invention

The current invention provides an alternative approach to treatment of prostate cancer that may be used alone or in combination with traditional treatment modalities.

One embodiment of the invention is a cancer vaccine comprised of genetically modified cells, typically cells which express a cytokine, such as GM-CSF, wherein following administration to a prostate cancer patient an enhanced immune response to beta-filamin protein is detected. In a further embodiment, the cells are of the same type as the tumor cells in the mammal. In general, the cancer vaccine is comprised of genetically modified tumor cells, however, non-tumor prostate cells also find utility in practicing the invention. The cells may be an established cell line that is grown and maintained in vitro. Established tumor cell lines for use in practicing the invention include, but are not limited to, PC-3 (ATCC#CRL-1435), Hela (ATCC#CCL-2), A549 (ATCC#CCL-185), LNCaP (ATCC#CRL-1740), H157 (ATCC#CRL-5802), or H1359 cells. In one approach, the genetically modified cells are derived from a tumor cell isolated from a subject and transduced with a vector that causes enhanced expression of a cytokine, e.g., GM-CSF. Then the genetically modified cells can be administered back to the same or a different subject as part of a cancer vaccine. It is understood that the descendants of a cell may not be completely identical (either morphologically, genotypically, or phenotypically) to the parent cell. Furthermore, the cells can be either an unselected population of cells or specific clones of cells. For example the cells can be genetically modified or screened for high expression levels of a cytokine such as GM-CSF or an antigen such as beta-filamin. The cells are typically human cells and in general, the cells cryopreserved prior to administration to the subject. Typically, the cells are proliferation-incompetent. In one embodiment, the cells are the progeny of a primary prostate tumor that has been established in ex vivo culture.

The degree of severity of prostate cancer is based on a variety of systems, one of which is disease staging, an example of which follows:

Stage 1: the cancer is very small and completely inside the prostate gland which feels normal when a rectal examination is done.

Stage 2: the cancer is still inside the prostate gland, but is larger and a lump or hard area can be felt when a rectal examination is done.

Stage 3: the cancer has broken through the covering of the prostate and may have grown into the neck of the bladder or the seminal vesicle.

Stage 4: the cancer has spread to another part of the body, where the most common site of prostate cancer spread is the bones. It does not often spread to other body organs.

Another criteria applied to evaluate the severity of prostate cancer disease is Gleason score, a different way of describing grade. When biopsies are taken, each area showing cancer cells is graded on a scale from one to five according to the appearance of the cells, with one as the lowest grade or most normal looking and five as the highest grade or least normal looking. A Gleason score is generated based on the average of two areas with the highest grade cells and adds their scores together to give the Gleason score.

Grade/Gleason only gives doctors an idea of how prostate cancer will progress and/or respond to treatment.

Pharmaceutical compositions comprising a cytokine-expressing cellular vaccine according to the invention may be used to treat a patient at any stage of prostate cancer, following, preceding, in lieu of, or in combination with, other therapies for treating prostate cancer in the subject. For example, the subject may previously or concurrently be treated by chemotherapy, external beam radiation therapy, and other forms of immunotherapy/cellular therapy, as further described below.

Treatment regimens for prostate cancer vary and include a range of treatment options including, but not limited to, one or more of surgery (i.e., radical prostatectomy); radiation therapy (i.e., external beam or brachytherapy); hormonal therapy, such as “androgen ablation”, e.g., administration of anti-androgens; and chemotherapy.

Anti-androgens most often used in the treatment of prostate cancer include, but are not limited to: leuprolide an injectable, synthetic hormone that is used to treat prostate cancer. Leuprolide (Lupron) is a gonadotropin-releasing hormone analog, which may be indicated for treatment of advanced prostate cancer. Leuprolide may be used in combination with one or both of Goserelin (Zoladex®) and Casodex (bicalutamide). Goserelin (Zoladex®) contains a synthetic decapeptide analogue of luteinizing hormone-releasing hormone (LHRH), also known as a gonadotropin releasing hormone (GNRH) agonist analogue. Casodex (bicalutamide) is an oral non-steroidal anti-androgen which contains the active ingredient bicalutamide. It works by blocking the effects of male hormones such as testosterone.

Flutamide is also used in the treatment of advanced prostate cancer. It works by preventing testosterone from binding to androgen receptors in the prostate gland. It also acts on an area of the brain called the hypothalamus, which ultimately results in a reduction in the amount of testosterone produced by the body. In the treatment of prostate cancer, flutamide is often used in combination with an LHRH analogue. LHRH analogues are one of the standard treatments for prostate cancer and include medicines such as buserelin, goserelin, leuprorelin and triptorelin.

Another drug used in the treatment of prostate cancer is Nilutamide (Anandron®), a nonsteroidal anti-androgen with affinity for androgen receptors (but not for progestogen, estrogen, or glucocorticoid receptors).

According to results presented at the 38th annual meeting of the American Society of Clinical Oncology and at the 40th annual meeting of the American Society of Clinical Oncology (ASCO), a treatment regimen containing Taxotere® (docetaxel), estramustine (Emcyt®) and prednisone was quite effective in treatment of hormone-refractory prostate cancer (HRPC).

Pharmaceutical compositions comprising a cytokine-expressing cellular vaccine according to the invention may be administered to a prostate cancer patient, sequential to, or in combination with, any currently used prostate cancer therapy, several examples of which are described above.

Typical means of monitoring prostate cancer in a subject, as generally known in the art, are carried out in conjunction with evaluation of the immune response to antigens which for which an enhanced immune response is detected following administration of a cytokine (e.g., GM-CSF) expressing cellular vaccine, according to the present invention. The patient may be monitored in any of a number of ways such as an evaluation of tumor mass, tumor volume, the number of tumor cells or growth rate of the tumor. Parameters that may be evaluated include but are not limited to, direct measurement of accessible tumors, counting of tumor cells (e.g. in the blood), measurement of tumor antigens (e.g., Prostate Specific Antigen (PSA), Alpha-fetoprotein (AFP), and the like), various visualization techniques (e.g., MRI, CAT-scan and X-rays), determination of bone density or evaluation of bone metastases. The information obtained from these analyses is useful in adjusting the dose or schedule of administration of the cellular vaccine in order to optimize the response of the individual and effect an improved therapeutic outcome relative to prostate cancer. Additional doses may be given as appropriate, typically on a biweekly basis, until the desired effect is achieved. Thereafter, additional booster or maintenance doses may be given as required. In a typical treatment regimen a cytokine (e.g., GM-CSF) expressing cellular vaccine is given as an intradermal injection (a needle placed directly under the skin) in the skin of the arms, legs or abdomen.

One aspect of the invention includes an assay for detecting an immune response to beta-filamin by obtaining serum samples from the prostate cancer patient prior to the first administration of the cytokine (e.g., GM-CSF) expressing cellular vaccine and at various time points following initiation of treatment. The amount of antibodies that bind beta-filamin from each time point is then compared. Any assay that quantitates the amount of anti beta-filamin antibody in a sample may be used for analyzing the serum for antibodies that bind beta-filamin. Examples of assays that can be used in the analysis of the serum for antibodies that bind beta-filamin include, but are not limited to, ELISA, Western blot, Immunofluorescence assay (IFA), FACS or Electrochemiluminescence (ECL). In one embodiment, the western blot uses a cell lysate of PC-3 cells as a source for the beta-filamin antigen.

In practicing the present invention, the cellular immune response to beta-filamin may be evaluated in the patient prior to the first administration of the cytokine (e.g., GM-CSF) expressing cellular vaccine and at various time points following initiation of treatment using antigen specific-cellular assays, proliferation assays, cytolytic cell assays, and in vivo delayed-type hypersensitivity testing with recombinant tumor-associated antigen or immunogenic fragments or peptides from the antigen. More methods to measure increased immune responses include assays currently used to measure T-cell responses such as delayed-type hypersensitivity testing, flow cytometry using peptide major histocompatibility complex tetramers, lymphoproliferation assay, enzyme-linked immunosorbant assay, enzyme-linked immunospot assay, cytokine flow cytometry, direct cytotoxicity assay, measurement of cytokine mRNA by quantitative reverse transcriptase polymerase chain reaction, and limiting dilution analysis. See, e.g., Lyerly HK, Semin Oncol. 2003 June; 30(3 Suppl 8):9-16.

The present invention is described by reference to the following Examples, which are offered by way of illustration and are not intended to limit the invention in any manner. Standard techniques well known in the art or the techniques specifically described below are utilized.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### EXAMPLES

### Example 1

### Generation of Recombinant Viral Vectors for Preparation of GVAX® Vaccines

The following viral vectors encoding a cytokine were constructed for introduction into tumor cell lines or into primary tumor cells obtained from resected human tumors.

(1) Retroviral Vectors

Construction of retroviral vectors employs standard ligation and restriction techniques, which are well understood in the art. A variety of retroviral vectors containing a gene or genes encoding a cytokine of interest were used. The MFG vector is described in U.S. Pat. Nos. 6,544,771 and 5,637,483. They are also described below with particular reference to the incorporation and expression of genes encoding cytokines. Furthermore, several MFG vectors have been deposited with the ATCC: the unmodified MFG vector was deposited as ATCC accession no. 68754; an MFG vector with a factor VIII insertion was deposited as ATCC accession no. 68726; and the MFG vector with a tPA insertion was deposited as ATCC accession no. 68727. The MFG vector is similar to the pEm vector, described below and in U.S. Pat. No. 6,544,771 and U.S. Pat. No. 5,637,483, but contains 1038 base pairs of the gag sequence for MoMuLV, to increase the encapsidation of recombinant genomes in the packaging cells lines, and 350 base pairs derived from MOV-9 which contains the splice acceptor sequence. An 18 base pair oligonucleotide containing Nco I and Bam HI sites directly follows the MOV-9 sequence and allows for the convenient insertion of genes with compatible sites. The coding region of the gene was introduced into the backbone of the MFG vector at the Nco I site and Bam HI site. The ATG initiator methionine codon was subcloned in frame into the Nco I site and little, if any, sequence beyond the stop codon was included, in order to avoid destabilizing the product and introducing cryptic sites. As a result, the ATG of the insert was present in the vector at the site at which the wild-type virus ATG occurs. Thus the splice was essentially the same as occurs in Moloney Murine Leukemia virus and the virus worked very well. The MoMuLV LTR controls transcription and the resulting mRNA contains the authentic 5′ untranslated region of the native gag transcript followed directly by the open reading frame of the inserted gene. In this vector, Moloney murine leukemia virus (Mo-MuLV) long terminal repeat sequences were used to generate both a full length viral RNA (for encapsidation into virus particles), and a subgenomic mRNA (analogous to the Mo-MuLV env mRNA) which is responsible for the expression of inserted sequences. The vector retained both sequences in the viral gag region shown to improve the encapsidation of viral RNA and the normal 5′ and 3′ splice sites necessary for the generation of the env mRNA. All oligonucleotide junctions were sequenced using the dideoxy termination method and T7 DNA polymerase. The virus is marker-free in that it does not comprise a dominant selectable marker (although one may optionally be inserted), and, given the high levels of transduction efficiency and expression inherent in the structure of the vector, transduction with MFG derivatives generally does not involve or require a selection step.

MFG vectors containing genes for the following proteins were constructed: murine IL-2, GM-CSF, IL-4, IL-5, gamma-IFN, IL-6, ICAM, CD2, TNF-alpha and IL-1-RA (interleukin-1-receptor antagonist). In addition, human sequences encoding TNF-alpha, GM-CSF and IL-2 were constructed, using publicly available sequence information. Precise cDNA sequences subcloned into MFG were as follows: murine IL-2 base pairs 49-564; murine IL-4 base pairs 56-479; murine IL-5 base pairs 44-462; murine GM-CSF (29) base pairs 70-561; murine ICAM-1 base pairs 30-1657; murine CD2 base pairs 48-1079; murine IL-1 receptor antagonist base pairs 16-563; human TNF-alpha base pairs 86-788.

(2) Adenoviral Vectors

An adenovirus vector for introduction of the coding sequence for human GM-CSF into cells (human GM-CSF; AV-GM-CSF) contains a GM-CSF expression cassette substituted for the E1 genes of adenovirus type 5 with an additional deletion in the viral genome in the E3 region. According to the complete GenBank sequence for Ad5 (Accession no. M73260), the deletions are from 455 to 3327 in the E1 region. Numbering begins with the first base of the left inverted terminal repeat.

Construction of the adenoviral vectors employs standard ligation and restriction techniques, which are well understood in the art. The E3 deletion was introduced by overlap recombination between wild type 300 (from H. Ginsberg) (0 to 27330) and d1324 (Thimmappaya et al. (1982) Cell 31:543-551) (21561 to the right end). The GM-CSF expression cassette was added to the E1 region by cre/lox mediated recombination between pAdlox MC hGM and E3 deleted adenovirus in CRE8 cells (Hardy et al. (1997) J. Virol. 71:1842-1949). pAdlox MD hGM was derived from pAdlox (Hardy et al. (1997) and pMD. G (Naldini et al, (1996) Science 272:263-267) and contains the following sequences: 0 to 455 from Ad5, the cytomegalovirus (CMV) immediate early gene promoter/enhancer (nucleotide positions-670 to +72, GenBank accession no. X03922) from pBC12/CMV/IL-2 (Cullen (1986) Cell 46: 973-982), a small region of human beta-globin exon 2 and a shortened second intervening sequence (IVS2) (nucleotide position 62613-62720 plus 63088-63532, GenBank accession no. J00179), exon 3 and the polyadenylation signal from human beta-globin (nucleotide positions 63532-64297), the GM-CSF cDNA inserted into exon 3 (position 63530), a second poly adenylation site from SV-40 (position 2681-2534), (GenBank accession no. J02400) and a loxP site followed by bacterial sequences from Bluescript. The GM-CSF cDNA was obtained from a plasmid (DNAX Research Institute of Molecular and Cellular Biology (Palo Alto, Calif.)). The DNA sequence was isolated from cDNA libraries prepared from Concanavalin A-activated human T-cell clones by functional expression in mammalian cells. The isolation and characterization of the cDNA, including the entire sequence of the gene, have been reported in the scientific literature. (Lee et al. (1985) Proc. Natl. Acad. Sci. (USA) 82:4360-4364). The identity of the clone was verified by restriction endonuclease digestion upon receipt. The MD expression cassette was modified by PCR to include restriction sites for PmlI, EcoRI and Bgl II for insertion of a transgene downstream of the IVS2. The GM-CSF cDNA was removed using PmlI and BamHI from pMFG-S hGM (Dranoff et al. (1997) Human Gene Ther. 7:111-123) and inserted into the PmlI and Bgl II sites in the MD cassette. The region of the pAdlox MD hGM plasmid that was incorporated into the adenovirus (Ad GM virus) was sequenced on both strands. Correct structure of the initial viral construction was confirmed by restriction analysis and ELISA testing for GM-CSF production from infected HeLa cells. The recombinant virus was then subjected to two rounds of plaque purification. Recombinant virus from a plaque was restriction mapped and expanded two passages by growth in 293 cells (certified cells from Microbiological Associates) to produce a research virus stock. The sequence of the GM-CSF gene in the virus was determined by direct sequencing of viral DNA prepared from the research virus stock. Finally, recombinant virus from the research virus stock was tested for mycoplasma and sterility, and when found negative, used to infect cells for the master virus stock.

### Example 2

### Autologous Prostate GVAX® Vaccine Trials

Nine patients were enrolled in an autologous prostate GVAX® vaccine trial. Each patient was greater than 18 years old with progressive, micrometastatic prostate cancer after surgery as defined by two successive abnormal elevations in PSA levels, without evidence of measurable metastatic disease or prior hormonal therapy, and with at least a baseline PSA of greater than 1.0 ng/ml at the start of treatment. The patients underwent surgery with appropriate concomitant medications. Pathologic diagnosis and staging of disease was completed during surgery.

A portion of the resected tumor was expanded in primary culture, transduced with the MFG viral vector carrying the GM-CSF gene, irradiated to render the cells proliferation-incompetent, and stored in liquid nitrogen until used to prepare the autologous GVAX® vaccine. Approximately 60 days after surgery, the vaccine was available for use at the clinical site. For each vaccination, the GVAX® vaccine was prepared and formulated for injection by thawing, washing, and resuspension of the cells in 0.9% sodium chloride solution or in 0.9% sodium chloride solution containing 2.5% human serum albumin.

Approximately 60 days after surgery, a prevaccination visit was scheduled to obtain baseline values and to initiate the first DTH evaluation of autologous, nontransduced cells. Serum was taken and PSA levels measured by RT-PCR. Two days after the prevaccination visit, each patient was scheduled through three vaccination cycles of 14 days each. If after three vaccinations there was no evidence of cumulative toxicity, and if sufficient vaccine cells remained, the patient was eligible to receive up to three additional vaccinations, for a total of six vaccinations.

The spacing and location of the vaccination sites are presented in Table 2 below. Each dose was administered to the patient on an outpatient basis, followed by clinical observation in the outpatient's department before discharge.

TABLE 2
VACCINATION SCHEDULE

| | | | Injection | |
|---|---|---|---|---|
| Dose Level | Dose Injected | Cells/ml | Volume | Injections |
| 1 | 1 × 10⁷ | 1 × 10⁷ | 0.5 cc | 2 |
| 2 | 5 × 10⁷ | 2.5 × 10⁷ | 0.5 cc | 5 |

Injections were given intradermally in the patients' limbs following a grid pattern. Each injection is at least 5 cm at needle entry from the nearest neighbor injection. For Dose Level 1, injections were given to three patients in one limb using a different limb in each successive cycle. For Dose Level 2, the injections in six patients were equally divided between two limbs in a given cycle, using two different limbs in each successive cycle. The first vaccination occurred on day 0 and subsequently on days 14, 28, 42, 56 and 70. Evaluation for local and systemic toxicities and induction of antitumor immune responses followed. Evidence of autoimmunity was also assessed.

No NCI CTC dose limiting toxicities were observed among eight vaccinated patients who received a total of 41 fully evaluable vaccinations. Biopsies of intradermal sites displayed distinctive inflammatory infiltrate, composed of macrophages, dendritic cells, eosinophils and T-cells similar to those observed in preclinical models of efficacy. 100% of patients displayed DTH reactivity to untransduced, autologous PCA target cells. The median serum PSA before surgery was 28.85 (with a range of 6.7-7.5) and the median PSA level at first vaccination was 0.65 (with a range of 0.1-30.4). By ultrasensitive serum PSA, 6/8 patients progressed after surgery and vaccination: average F/U 24 months. This study demonstrates the feasibility, outpatient safety, and bioactivity of in vivo GM-CSF gene-transduced PCA vaccines.

### Example 3

### Allogeneic Prostate GVAX® Vaccine Trial

Thirty patients were enrolled in a second autologous prostate GVAX® vaccine trial. Each patient was greater than 18 years old with progressive, micrometastatic prostate cancer after surgery as defined by two successive abnormal elevations in PSA levels, without evidence of measurable metastatic disease or prior hormonal therapy, and with at least a baseline PSA of greater than 1.0 ng/ml at the start of treatment. The allogeneic prostate cancer cell line vaccine is composed of two equal cell doses of allogeneic prostate cancer cell lines (LNCaP and PC-3) genetically modified to secrete 148-639 ng of GM-CSF/10⁶ cells/24 hours. Alternatively, the vaccine is composed of a mixture of three different irradiated, autologous prostate cancer cell lines (LNCaP, PC-3 and DU 145) genetically modified to secrete 200-300 ng of GM-CSF/10⁶ cells/24 hours. Each vial of vaccine is prepared as a direct injectable in glycerol and human serum albumin. The dose of each cell line vaccination is presented in Table 3, below.

TABLE 3
VACCINATION SCHEDULE

| | Dose | Cells/ | Injection | |
|---|---|---|---|---|
| CellLIne | Injected | ml | Volume | Injections |
| LNCaP-1740 | 6 × 10⁷ | 3 × 10⁷ | 2 × 1.0 cc | 2 of 3 × 10⁷ cells |
| PC-3 | 6 × 10⁷ | 3 × 10⁷ | 2 × 1.0 cc | 2 of 3 × 10⁷ cells |

On a given vaccination day, the patient received a total of 1.2×10⁸ total cells (6×10⁷ cells per cell line), given in 4 intradermal injections of 1.0 cc each (2 injections per cell line). Each injection was intradermal. On vaccination day subsequent to Day 0, the injection sites were rotated. A total dose of 1.2×10⁸ cells, divided into 4 injections, is being given once every week for 8 weeks.

During the treatment cycle, evaluations for local systemic reaction to the vaccination were performed on the day of vaccination (Week 1, 2, 3, 4, 5, 6, 7 and 8). Starting from the first vaccination,

PSA measurements were determined every month for 4 months, and then every 4 months for two years in Part 2 of the study. PSA levels were evaluated more frequently than every 4 months if clinically indicated. A blood sample for PCR testing was drawn prior to the first vaccination. The final visit for Part 1 occurs two weeks after administration of the last vaccination (Week 8). Enrolled patients who received at least one vaccination participated in long-term follow-up evaluations with their PSA checks. They had yearly physical examinations and clinical evaluations thereafter, or more frequently as clinically indicated until the patient died or until allogeneic prostate cancer cell line vaccines are approved by the FDA.

### Example 4

### Identification of Prostate Tumor-Associated Antigens

A. Preparation of Sera

Sera used for the studies were prepared from the blood drawn from the patients in autologous or allogeneic prostate GVAX® vaccine trials two hours before vaccination and two weeks after final vaccination.

B. Preparation of Cell Lysates

Primary cell lines derived from prostate stromal, prostate epithelia, prostate smooth muscle, and lung fibroblast, were purchased from Clontenics (San Diego, Calif.) and were grown in SCGM, PrEGM, SmGM, and FGM-2 medium (Clontenics, San Diego, Calif.). Cells were grown in the DMEM+F12 medium (JHR bioscience, Lenexa, Kans.) containing 10% fetal calf serum, penicillium/strepavidin, and glutamine. When cell density reached 80% confluence in T-175 flasks (Becton, Dickinson & Company, Franklin Lakes, N.J.), cells were washed two times with PBS followed by incubation in Versene (Gibco BRL, Grand Island, N.Y.) for 10-30 minutes to detach the cells from the flasks. The cells were then harvested and spun down in table-top centrifuge (CS-6R, Beckman, Palo Alto, Calif.) at 1,000 rpm for 10 minutes. Cells were washed three times with PBS. For non-adherent cells (Jurkat and peripheral blood cells), cells were harvested, spun down, and washed three times with PBS. After washing, 2×10⁷ cells were lysed with 1 ml of lysis buffer (10 mM Tris pH 7.4, 1 mM EDTA, 10% glycerol, 1% NP40, 1 mM PMSF, and 1% protease inhibitor cocktail set III (Cat. 539134, Calbiochem, San Diego, Calif.)), followed by incubation on ice for one hour. Insoluble cell debris was then removed by centrifugation using a table-top eppendorf centrifuge at 4° C. for 30 minutes. The supernatant was removed and the protein concentration measured by BCA (Pierce, Rockford, Ill.).

C. Western Blot Analysis

Indicated amounts of protein (25-35 ug/lane) in cell lysates or purified PSA (Calbiochem, San Diego, Calif.) or other cancer-associated markers were separated on a 4-20% gradient SDS-PAGE (Norvex, San Diego, Calif.), followed by electro-transferring to a nitrocellulose membrane (Norvex, San Diego, Calif.) in a transblot apparatus (Xcell II, Blot Module, Norvex, San Diego, Calif.) at 25 mV constant voltage for 2-3 hours. After transferring, the nitrocellulose membrane was blotted with blocking solution (10% non-fat milk in 0.05% Tween 20 in PBS) overnight at 4° C. After overnight blocking, the membrane was incubated with patient's serum (1:1000 dilution in PBS+0.05% Tween 20) at room temperature for 2 hours followed by five washes with PBS+0.1% Tween 20. HPRT-conjugated goat anti-human IgM+G+A (Zymed, South San Francisco, Calif., 1:3000 dilution in PBS+0.05% Tween 20) was incubated with membrane for one hour followed by six washes with PBS+0.1% Tween 20. The results were developed by chemifluorescence (e.g., using the ECL Western blotting system, Amersham Life Science, Arlington Heights, Ill.).

### Example 5

### Prostate Tumor-Associated Antigens Identified in Autologous GVAX® Clinical Trials

In one study, carcinoma-related antigens were identified as follows. To prepare autologous prostate GVAX® vaccine, prostate tumors were removed from eight patients to generate primary prostate cancer cell lines. Human GM-CSF containing retroviral vectors were transduced into these primary cell lines to generate cells secreting GM-CSF. Patients were administered a vaccine in the form of these GM-CSF expressing autologous primary prostate cancer cells every two weeks by intradermal injection. Patients 1, 2, and 3 received 1×10⁷ cells for six administrations; patient 4 received 1×10⁷ cells for five administrations; patient 5 and 7 received 5×10⁷ cells for six administrations; patient 7 received 1×10⁷ cells for three administrations; and patient 8 received 5×10⁷ cells for three administrations. Sera used for the following studies were prepared from blood taken two hours before vaccination (as pre-vaccination) and two weeks after final vaccination (as post-vaccination).

Identification of the specific antigens recognized by the antibodies in the sera of the patients in autologous prostate GVAX® vaccine trials after final vaccination was made by Western blot analysis of the LNCaP prostate cancer cell line. 25 ug of LNCaP lysate was run on the 4-20% gradient SDS polyacrylamide gels, followed by transference to a nitrocellulose membrane. A dilution in the range of 1:1000 to 1:3000 of patients' sera in PBS containing 0.05% Tween 20 was used for the primary antibody in the Western blot analysis. A dilution of 1:3000 of peroxidase-conjugated polyclonal goat anti-human IgG+M+A was used for the secondary antibody. The results were developed by chemifluorescence ECL kit. A variety of antigens unrelated to PSA and expressed by LNCaP cells were newly identified by comparing the sera derived from pre-and post-vaccination in an autologous GVAX® vaccine trial including a “pan” tumor-associated antigen, as further described in WO/0026676, expressly incorporated by reference herein.

### Example 6

### Prostate Tumor-Associated Antigens Identified in Allogeneic GVAX® Clinical Trials

Novel antigens were also identified by the sera of patients treated with an allogeneic GVAX® vaccine. In the allogeneic prostate GVAX® vaccine trial, 21 patients were vaccinated with both 1.2×10⁷ GM-CSF-expressing LNCaP (LNCaP/GM) cells and GM-CSF expressing (PC-3/GM) cells weekly for eight weeks. Sera were prepared from blood taken two hours before GVAX® vaccine administration (as “pre-vaccination”) and two weeks after final GVAX® vaccine administration (as “post-vaccination”). The sera derived from several allogeneic GVAX® vaccine-treated patients were selected for further study. This data is summarized in Table 4, below:

TABLE 4

| Patient | PC3 Antigen | LNCaP Antigen |
|---|---|---|
| 301 | p14, p18, p27 | |
| 302 | | |
| 303 | | |
| 304 | | |
| 305 | p14, p160, p278, p300 | |
| 306 | p12, p32, p45, p80, p105 | |
| 307 | p32, P43 | |
| 308 | p18 | p40, p55, p68 |
| 309 | p19, p27 | p19 |
| 310 | p278, p300 | |
| 311 | | |
| 312 | | p42, p112 |
| 313 | p70 | |
| 314 | p14, p60, p130 | |
| 315 | p14, p23, p27 | |
| 316 | p278, p300 | |
| 317 | | |
| 318 | | |
| 319 | p29, p43, p60 | |
| 320 | | p150 |
| 321 | p278, p300 | |

Humoral immune responses against LNCaP and/or PC3 cells were observed in the majority of cases. The induction of such anti-PC3 and anti-LNCaP antibody responses, and the observed decrease in PSA velocity in 15 of 21 allogeneic GVAX® vaccine-treated prostate cancer patients, suggests that the humoral immune response plays a therapeutic role in treatment of prostate tumors in the setting of an allogeneic GVAX®. It the ability of sera from post-allogeneic GVAX® vaccine treated patients was then examined for antibodies to PSA. 3 ug of PSA was analyzed by 4-20% gradient SDS-PAGE followed by Western analysis using the sera from patients 301, 305, 314, 307, and 312. The results show that PSA is not recognized by these sera, indicating PSA cannot be responsible for eliciting the corresponding immune response to eradicate tumor growth. To further characterize these novel antigens, Western blot analysis was carried out using pre-and post-vaccination sera derived from a number of patients. The following panel of carcinoma cell lines were examined: LNCaP (prostate); PC-3 (prostate); A549 (lung); LS-174T (colon); MCF7 (breast); DU-145 (prostate); KLEB (ovarian); Jurkat (leukemia); and MDA-MB-435S (breast). The tissue/cell-specific expression of an approximately 278 kD and an approximately 160 kD antigen, as determined by SDS-PAGE, was characterized using this panel of carcinoma cell lines and normal primary prostate cell lines. The approximately 160 kD antigen was shown to be expressed on PC-3 cancer and normal prostate epithelial cells, weakly on A549 (lung carcinoma) and MCF7 (breast carcinoma), but not on other types of carcinoma tested, nor on prostate stromal or smooth muscle cells. These results indicate that the approximately 160 kD antigen (p160), is a prostate-specific antigen (data not shown). From the same experiments, tumor-associated antigen having a molecular weight of approximately 278 kD as determined by SDS-PAGE (p278) was found to be expressed on prostate cancer cell lines, PC-3 and DU-145, and A549 lung carcinoma. p278 was also shown to be expressed on normal prostate epithelial cells but not on stromal or smooth muscle cells. These results strongly indicate that p278, is at least a prostate-specific antigen and a tumor-associated antigen. The fact that there is an antibody response to normal prostate-specific antigens, such as p278 and p160, indicates that the allogeneic GVAX® vaccines can break tolerance and cause the immune system to mount a response against such tumors by recognizing tumor-associated antigens.

### Example 7

### Prostate Tumor-Associated Antigens Identified in Phase II Allogeneic GVAX® Clinical Trials

Other novel antigens were also identified by the sera of patients treated with an allogeneic GVAX® vaccine in a further clinical trial. In this allogeneic prostate GVAX® vaccine trial, 24 hormone-refractory patients with metastatic bone disease received a 500 million cell prime dose of GM-CSF-expressing LNCaP cells and GM-CSF expressing PC-3 cells followed by 12 booster doses (100 million cells each) at 2-week intervals, and 10 patients received the same prime dose and a higher booster dose of 300 million cells. Sera were prepared from blood taken two hours before GVAX® vaccine administration (as “pre-vaccination”) and two weeks after final GVAX® vaccine administration (as “post-vaccination”). The sera derived from patients were further studied. Some of the antigens recognized post-vaccination following SDS-PAGE and Western blot are summarized in Table 5, below:

TABLE 5

| Patient | PC3 Antigen | LNCaP Antigen |
|---|---|---|
| GO3-009-307T | p20 | p70 |
| GO3-009-315HH | | |
| GO3-009-322W | p50 | p50, p70 |
| GO3-012-401D | p278 | p40 |
| GO3-012-402E | p278, p180, p150, p10 | |
| GO3-012-406H | p70 | p20 |
| GO3-012-407G | p40 | |
| GO3-012-414L | p180, p160, p150, p60, p10 | p60 |
| GO3-012-417R | | |
| GO3-012-418W | p200, p180, p160 | |
| GO3-012-421Y | | p50, p20 |
| GO3-012-422V | p278, p110, p10 | |
| GO3-014-423S | p100, p90 | p120, p150 |
| GO3-012-428RR | | |
| GO3-012-429GG | | |
| GO3-012-431QQ | p70 | |
| GO3-012-436TT | | |
| GO3-014-101D | | |
| GO3-014-102JJ | p160 | p130 |
| GO3-014-103MM | p30, p20 | |
| GO3-014-104VV | | |
| GO3-018-802EE | p30, p20 | |
| GO3-018-803II | p110, p100 | |
| GO3-018-804SS | p278 | |
| GO3-018-806NN | | |
| GO3-009-310BB | | p20 |
| GO3-009-314LL | p10 | p50, p30, p20 |
| GO3-009-320OO | | |

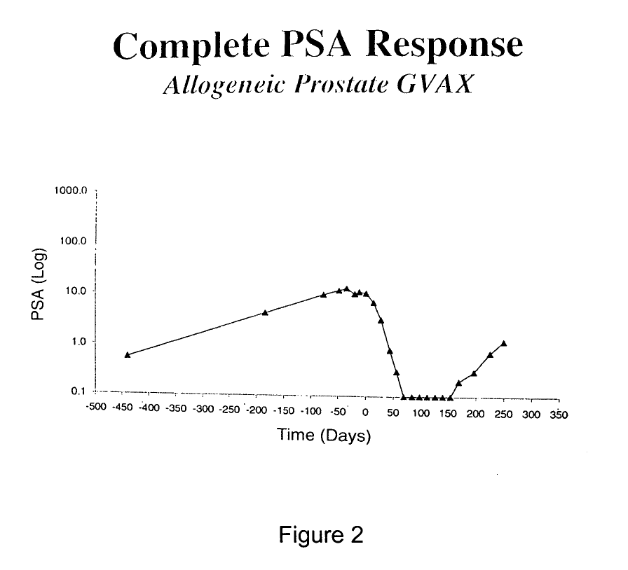

Humoral immune responses against LNCaP and/or PC3 cells were observed in the majority cases. Post-vaccination sera did not contain any anti-PSA antibodies. A complete PSA response was observed for patient 804 (FIG. 2). There was no HLA Class I type match between patient 804 and either the PC-3 or LNCaP cell types.

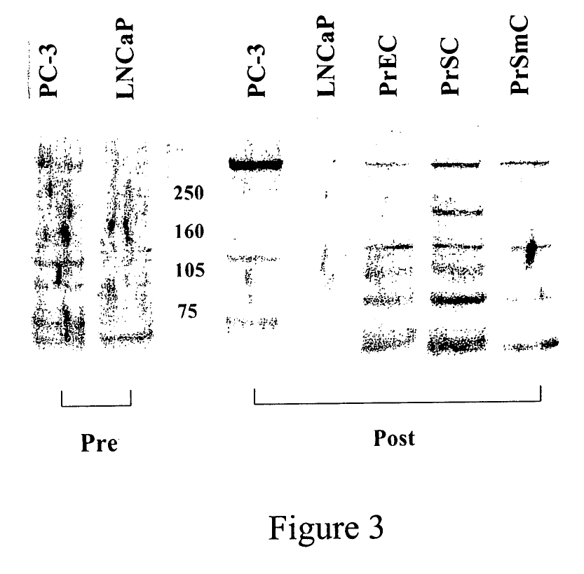

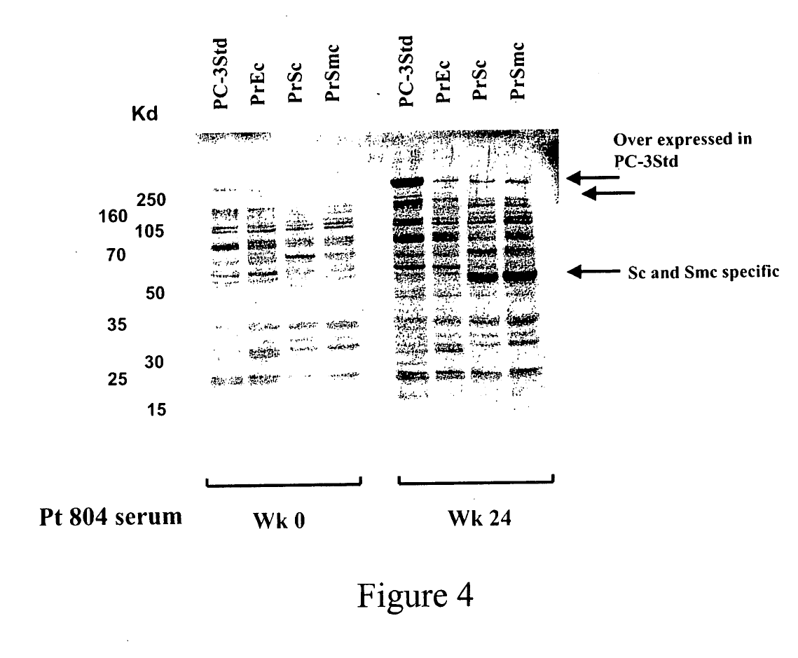

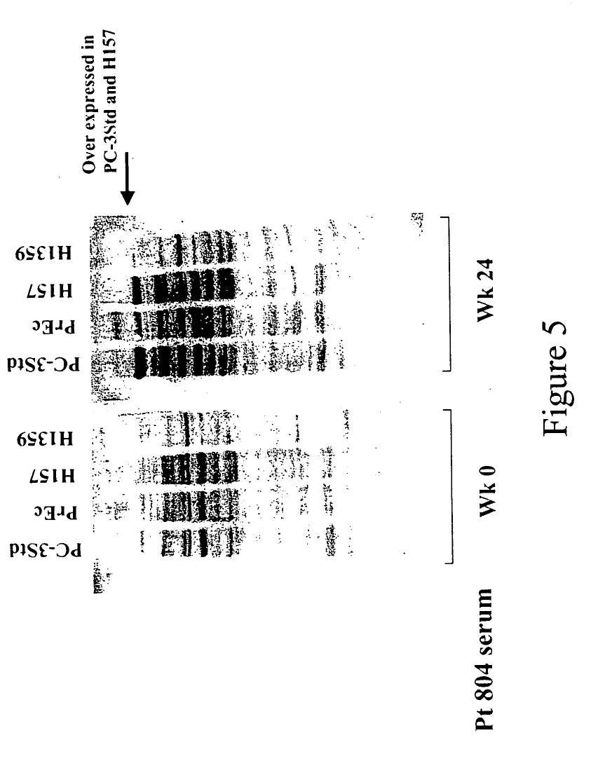

To characterize the humoral response of patient 804 to GVAX® treatment, pre-and post-vaccination sera from patient 804 was used in Western blot analysis. Post-vaccination serum was found to recognize an antigen migrating at approximately 278 kD that is present in PC-3 but not LNCaP cells (FIG. 3). The approximately 278 kD antigen was also detected in primary normal prostate epithelial, prostate stromal, and prostate smooth muscle (PrEC, PrSC, and PrSmC) cell lines but at lower levels than in PC-3 cells (FIG. 4). The antigen is also overexpressed in non-small cell lung cancer cell line, H157 (FIG. 5).

PC-3 cell lysates were subject to two dimensional gel electrophoresis, with a pH gradient along the horizontal dimension of 3.5-10. Migration of the approximately 278 kD antigen was determined by blot analysis. The relevant band was excised and subjected to MALDI-MS protein sequencing. The protein sequence of the approximately 278 kD antigen matches the sequence of beta filamin found at GenBank Accession # NP_{—}001448, an actin-binding protein.

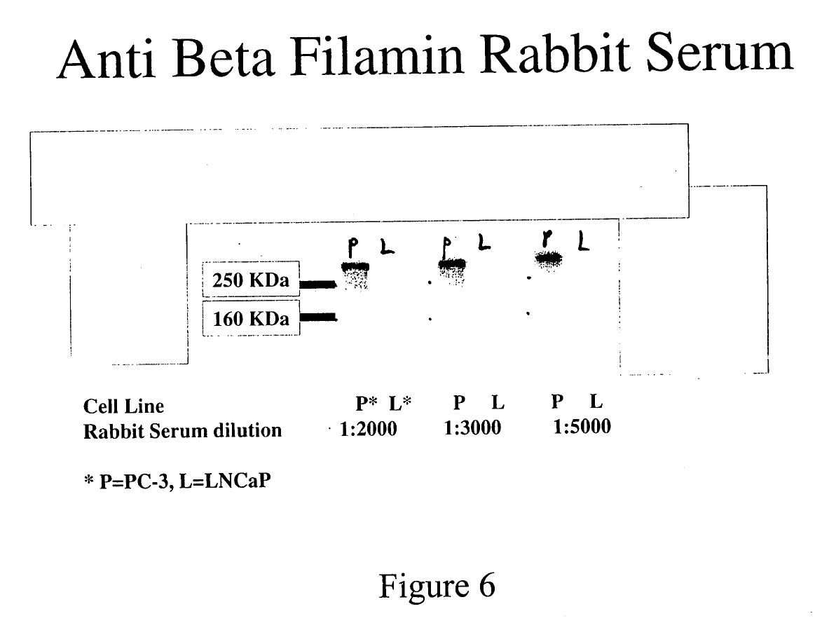

Rabbit anti-human filamin monoclonal antibodies from Chemicon International (Temecula, Calif.) were demonstrated to react with the approximately 278 kD protein expressed in PC-3 but not in LNCaP cells by Western blot (FIG. 6).

### Example 8

### Vaccination with Allogeneic Cells Expressing Beta Filamin and GM-CSF

Retroviral vectors are prepared by inserting the coding sequence for GM-CSF together with the coding region for beta filamin shown in described in GenBank Accession # NM_{—}001457 using standard techniques as described in WO/0026676. PC-3 and LNCaP cells are transduced and genetically modified to express beta filamin, and PC-3 cells are genetically altered with such that they express higher levels of beta filamin than untransduced PC-3 cells. Transduced PC-3 and LNCaP cells are mixed at a 1:1 ratio to form a cellular vaccine and the cells are rendered proliferation incompetent by irradiation. 200 million cells of either vaccine secretes about 100 ug GM-CSF per 10⁶ cells per day.

Patients were selected who have adenocarcinoma of the prostate, rising PSA, ECOG performances status 0 to 1, normal liver, renal and bone marrow function, no previous chemotherapy or gene therapy, no active autoimmune disease, no concomitant treatment for prostate cancer, and metastatic disease with positive bone scan but without bone pain requiring narcotic analgesics. Group A of patients is treated with vaccine A, and group B of patients is treated with vaccine B. For each group, a prime dose of 500 million cells is followed by 12 booster doses of 100 or 200 million cells at two-week intervals. Sera is prepared from blood taken from each patient two hours before the first vaccine administration, and two weeks after the final vaccine administration.

Response is monitored by time to disease progression as measured by bone scan, bone density evaluation, CT scan, PSA level, and survival.

Brief Description of the Sequences

TABLE 6
Table of Sequences

| | |
|---|---|
| SEQ ID NO | SEQUENCE |

| | |
|---|---|
| 1 | nucleic acid coding sequence for GM-CSF (genomic): |
| | TGTGGCTGCAGAGCCTGCTGCTCTTGGGCACTGTGGCCTGCAGCATCTCTGCACCCGCCCGCTCGCC |
| | CAGCCCCAGCACGCAGCCCTGGGAGCATGTGAATGCCATCCAGGAGGCCCGGCGTCTCCTGAACCTG |
| | AGTAGAGACACTGCTGCTGAGATGGTAAGTGAGAGAATGTGGGCCTGTGCCTAGGCCACCCAGCTGG |
| | CCCCTGACTGGCCACGCCTGTCAGCTTGATAACATGACATTTTCCTTTTCTACAGAATGAAACAGTAGAA |
| | GTCATCTCAGAAATGTTTGACCTCCAGGTAAGATGCTTCTCTCTGACATAGCTTTCCAGAAGCCCCTGC |
| | CCTGGGGTGGAGGTGGGGACTCCATTTTAGATGGCACCACACAGGGTTGTCCACTTTCTCTCCAGTCA |
| | GCTGGCTGCAGGAGGAGGGGGTAGCAACTGGGTGCTCAAGAGGCTGCTGGCCGTGCCCCTATGGCA |
| | GTCACATGAGCTCCTTTATCAGCTGAGCGGCCATGGGCAGACCTAGCATTCAATGGCCAGGAGTCACC |
| | AGGGGACAGGTGGTAAAGTGGGGGTCACTTCATGAGACAGGAGCTGTGGGTTTGGGGCGCTCACTGT |
| | GCCCCGAGACCAAGTCCTGTTGAGACAGTGCTGACTACAGAGAGGCACAGAGGGGTTTCAGGAACAAC |
| | CCTTGCCCACCCAGCAGGTCCAGGTGAGGCCCCACCCCCCTCTCCCTGAATGATGGGGTGAGAGTCA |
| | CCTCCTTCCCTAAGGCTGGGCTCCTCTCCAGGTGCCGCTGAGGGTGGCCTGGGCGGGGCAGTGAGAA |
| | GGGCAGGTTCGTGCCTGCCATGGACAGGGCAGGGTCTATGACTGGACCCAGCCTGTGCCCCTCCCAA |
| | GCCCTACTCCTGGGGGCTGGGGGCAGCAGCAAAAAGGAGTGGTGGAGAGTTCTTGTACCACTGTGGG |
| | CACTTGGCCACTGCTCACCGACGAACGACATTTTCCACAGGAGCCGACCTGCCTACAGACCCGCCTGG |
| | AGCTGTACAAGCAGGGCCTGCGGGGCAGCCTCACCAAGCTCAAGGGCCCCTTGACCATGATGGCCAG |
| | CCACTACAAGCAGCACTGCCCTCCAACCCCGGTGAGTGCCTACGGCAGGGCCTCCAGCAGGAATGTC |
| | TTAATCTAGGGGGTGGGGTCGACATGGGGAGAGATCTATGGCTGTGGCTGTTCAGGACCCCAGGGGG |
| | TTTCTGTGCCAACAGTTATGTAATGATTAGCCCTCCAGAGAGGAGGCAGACAGCCCATTVCATCCCAAG |
| | GAGTCAGAGCCACAGAGCGCTGAAGCCCACAGTGCTCCCCAGCAGGAGCTGCTCCTATCCTGGTCATT |
| | ATTGTCATTATGGTTAATGAGGTCAGAGGTGAGGGCAAACCCAAGGAAACTTGGGGCCTGCCCAAGGC |
| | CCAGAGGAAGTGCCCAGGCCCAAGTGCCACCTTCTGGCAGGACTTTCCTCTGGCCCCACATGGGGTG |
| | CTTGAATTGCAGAGGATCAAGGAAGGGGGGCTACTTGGAATGGACAAGGACCTCAGGCACTCCTTCCT |
| | GCGGGAAGGGAGCAAAGTTTGTGGCCTTGACTCCACTCCTTCTGGGTGCCCAGAGACGACCTCAGCC |
| | CAGCTGCCCTGCTCTGCCCTGGGACCAAAAAGGCAGGCGTTTGACTGCCCAGAAGGCCAACCTCAGG |
| | CTGGCACTTAAGTCAGGCCCTTGACTCTGGCTGCCACTGGCAGAGCTATGCACTCCTTGGGGAACACG |
| | TGGGTGGCAGCAGCGTCACCTGACCCAGGTCAGTGGGTGTGTCCTGGAGTGGGCCTCCTGGCCTCTG |
| | AGTTCTAAGAGGCAGTAGAGAAACATGCTGGTGCTTCCTTCCCCCACGTTACCCACUGCCTGGACTCA |
| | AGTGTTTTTTATTTTTCTTTTTTTAAAGGAAACTTCCTGTGCAACCCAGATTATCACCTTTGAAAGTTTCAA |
| | AGAGAACCTGAAGGACTTTCTGCTTGTCATCCCCTTTGACTGCTGGGAGCCAGTCCAGGAGTG |
| 2 | nucleic acid coding sequence for GM-CSF (cDNA): |
| | ATGTGGCTGCAGAGCCTGCTGCTCTTGGGCACTGTGGCCTGCAGCATCTGTGTGCACCCGCCCGCTCGC |
| | CCAGCCCCAGCACGCAGCCCTGGGAGCATGTGAATGCCATCCAGGAGGCCCGGCGTCTCCTGAACCT |
| | GAGTAGAGACACTGCTGCTGAGATGAATGAAACAGTAGAAGTCATCTCAGAAATGTTTGACCTCCAGGA |
| | GCCGACCTGCCTACAGACCCGCCTGGAGCTGTACAAGCAGGGCCTGCGGGGCAGCCTCACCAAGCTC |
| | AAGGGCCCCTTGACCATGATGGCCAGCCACTACAAGCAGCACTGCCCTCCAACCCCGGAAACTTCCTG |
| | TGCAACCCAGACTATCACCTTTGAAAGTTTCAAAGAGAACCTGAAGGACTTTCTGCTTGTCATCCCCTTT |
| | GACTGCTGGGAGCCAGTCCAGGAGTAA |
| 3 | Amino acid sequence for GM-CSF |
| | MWLQSLLLLGTVACSISAPARSPSPSTQPWEHVNAIQEARRLLNLSRDTAAEMNETVEVISEMFDLQEPTCL |
| | QTRLELYKQGLRGSLTKLKGPLTMMASHYKQHCPPTPETSCATQTITFESFKENLKDFLLVIPFDCWEPVQE |
| 4 | Filamin B 9432 bp mRNA sequence including coding region: GenBank |
| | Accession # NM_001457 |
| 5 | 2602 amino acid sequence for Filamin B: GenBank |
| | Accession # NP_001448 |

Although the foregoing invention has been described in some detail by way of illustration and example for purposes of clarity and understanding, it will be apparent to those skilled in the art that certain changes and modifications may be practiced. Various aspects of the invention have been achieved by a series of experiments, some of which are described by way of the following non-limiting examples. Therefore, the description and examples should not be construed as limiting the scope of the invention, which is delineated by the appended claims.

1. A method of treating prostate cancer in a subject, comprising:
(a) genetically modifying a first population of tumor cells to produce GM-CSF;
(b) administering said tumor cells to a subject;
(c) detecting an immune response to an approximately 278 kD antigen as determined by SDS-PAGE,
wherein said immune response is not detected prior to said administering.

2. The method according to claim 1, wherein said approximately 278 kD antigen is beta filamin.

3. The method according to claim 2, wherein said immune response is a humoral immune response.

4. The method according to claim 2, wherein said first population of tumor cells is proliferation-incompetent.

5. The method according to claim 4, wherein said first population of tumor cells are allogeneic.

6. The method according to claim 4, wherein said first population of tumor cells are autologous.

7. The method according to claim 4, wherein said first population of tumor cells are bystander cells.

8. The method according to claim 5, wherein said first population of tumor cells are PC-3 cells or LNCaP cells.

9. The method according to claim 8, wherein said immune response is a humoral immune response.

10. The method according to claim 4, wherein a second population of tumor cells is genetically modified to produce GM-CSF and co-administered with said first population of tumor cells.

11. The method according to claim 10, wherein genetically modified cells are PC-3 cells and LNCaP cells.

12. A method of treating prostate cancer in a subject, comprising:
genetically modifying a first population of tumor cells to produce GM-CSF;
combining said first population of tumor cells with a second population of tumor cells;
administering said first and second populations of tumor cells to a subject;
detecting an immune response to an approximately 278 kD antigen as determined by SDS-PAGE,
wherein said immune response is not detected prior to said administering.

13. The method according to claim 10, wherein said approximately 278 kD antigen is beta filamin.

14. The method according to claim 11, wherein said immune response is a humoral immune response.

15. The method according to claim 11, wherein said first population of tumor cells is proliferation-incompetent.

16. The method according to claim 13, wherein said first population of tumor cells are allogeneic.

17. The method according to claim 13, wherein said first population of tumor cells are autologous.

18. The method according to claim 13, wherein said first population of tumor cells are bystander cells.

19. The method according to claim 11, wherein said second population of tumor cells is proliferation-incompetent.

20. The method according to claim 17, wherein said second population of tumor cells are allogeneic.

21. The method according to claim 17, wherein said second population of tumor cells are autologous.

22. The method according to claim 17, wherein said second population of tumor cells are bystander cells.

23. The method according to claim 14, wherein said first population of tumor cells is selected from the group consisting of PC-3 cells, LNCaP cells, and PC-3 cells plus LNCaP cells.

24. The method according to claim 21, wherein said immune response is a humoral immune response.

25. A method of reducing the level of PSA in a prostate cancer patient, said method comprising:
genetically modifying a first population of tumor cells to produce GM-CSF;
administering said cells to a subject;
detecting an immune response to an approximately 278 kD antigen as determined by SDS-PAGE, wherein said immune response is not detected prior to said administering.

26. The method according to claim 23, wherein said approximately 278 kD antigen is beta filamin.

27. The method according to claim 24, wherein said immune response is a humoral immune response.

28. The method according to claim 24, wherein said first population of tumor cells is proliferation-incompetent.

29. The method according to claim 26, wherein said first population of tumor cells are allogeneic.

30. The method according to claim 26, wherein said first population of tumor cells are autologous.

31. The method according to claim 26, wherein said first population of tumor cells are bystander cells.

32. The method according to claim 27, wherein said first population of tumor cells is selected from the group consisting of PC-3 cells, LNCaP cells, and PC-3 cells plus LNCaP cells.

33. The method according to claim 30, wherein said immune response is a humoral immune response.

34. A method of reducing the level of PSA in a prostate cancer patient, said method comprising:
genetically modifying a first population of tumor cells to produce GM-CSF;
combining said first population of tumor cells with a second population of tumor cells;
administering said first and second populations of tumor cells to a subject;
detecting an immune response to an approximately 278 kD antigen as determined by SDS-PAGE, wherein said immune response is not detected prior to said administering.

35. The method according to claim 32, wherein said approximately 278 kD antigen is beta filamin.

36. The method according to claim 33, wherein said immune response is a humoral immune response.

37. The method according to claim 33, wherein said first population of tumor cells is proliferation-incompetent.

38. The method according to claim 35, wherein said first population of tumor cells are allogeneic.

39. The method according to claim 35, wherein said first population of tumor cells are autologous.

40. The method according to claim 35, wherein said first population of tumor cells are bystander cells.

41. The method according to claim 33, wherein said second population of tumor cells is proliferation-incompetent.

42. The method according to claim 39, wherein said second population of tumor cells are allogeneic.

43. The method according to claim 39, wherein said second population of tumor cells are autologous.

44. The method according to claim 39, wherein said second population of tumor cells are bystander cells.

45. The method according to claim 36, wherein said first population of tumor cells is selected from the group consisting of PC-3 cells, LNCaP cells, and PC-3 cells plus LNCaP cells.

46. The method according to claim 43, wherein said immune response is a humoral immune response.
